(19) United States
(12) Patent Application Publication
(10) Pub. No.: US 2008/0049319 A1
(43) Pub. Date: Feb. 28, 2008

(54) POLYMERIZABLE MONOMER, AN OPTICAL COMPENSATION FILM AND A METHOD FOR PRODUCING THE OPTICAL COMPENSATION FILM

(75) Inventors: Takahiro KATO, Minami-ashigara-shi (JP); Yi Li, Minami-ashigara-shi (JP); Naoyuki Nishikawa, Minami-ashigara-shi (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Tokyo (JP)

(21) Appl. No.: 11/843,388

(22) Filed: Aug. 22, 2007

(30) Foreign Application Priority Data
Aug. 23, 2006 (JP) 2006-226888

(51) Int. Cl. G02B 5/30; B05D 3/00; C07C 69/76; G02F 1/1335; C08F 18/02

(52) U.S. Cl. 359/485; 349/117; 427/385.5; 526/326; 560/85

(57) ABSTRACT

A polymerizable monomer represented by the following Formula (I): Formula (I)

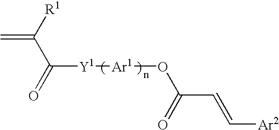

wherein R¹ represents a hydrogen atom or a substituted group; Y¹ represents an oxygen atom or —NR³—, wherein R³ represents a hydrogen atom or an alkyl group; Ar¹ and Ar² each independently represents an aromatic ring having from 1 to 10 carbon atoms, and each of Ar¹ and Ar² may have a substituted group; and n represents an integer of from 1 to 3.

### FIELD OF THE INVENTION

The present invention relates to a polymerizable monomer, a high molecular compound, an optically anisotropic film, an optically compensatory film, a polarizing plate and a liquid crystal display device, and a method for producing an optically compensatory film. More specifically, the invention relates to a novel polymerizable monomer and a high molecular compound from which an optically anisotropic film having an optical characteristic which is in general difficultly prepared can be obtained, and an optically anisotropic film, an optically compensatory film, a polarizing plate and a liquid crystal display device each using these, and a process for producing an optically compensatory film.

### BACKGROUND OF THE INVENTION

As an image display device which is used for OA appliances such as word processors, notebook personal computers and monitors for personal computers, mobile terminals, television set and the like, a Cathode Ray Tube (CRT) has been mainly used until now. In recent years, a liquid crystal display device is being widely used in place of CRT because it is slim, lightweight and low in electric power consumption. The liquid crystal display device is provided with a liquid crystal cell and a polarizing plate. The polarizing plate is in general composed of a protective film and a polarizing film and obtained by dyeing a polarizing film composed of a polyvinyl alcohol film with iodine, stretching it and stacking both surfaces thereof by a protective film. Also, for the purpose of realizing enhancement in the contrast of an image display device and enlargement in the viewing angle thereof, in most cases, an optically compensatory film and a retardation film are used. As such optically compensatory film and retardation film, a stretched film having refractive index anisotropy and a film obtained by orienting a liquid crystalline compound and polymerizing it are used.

In recent years, with respect to these optically compensatory film and retardation film to be used for image display devices, for the purpose of further enhancing a viewing angle characteristic and contrast of an image display device, it is required to control more precisely the refractive index anisotropy. Under these present circumstances, the foregoing stretched film involves a problem that not only the stretch direction at the manufacture is limited, but it is difficult to control precisely the refractive index anisotropy. On the other hand, in the film obtained by orienting a liquid crystalline compound and polymerizing it, the liquid crystalline compound is in general oriented by subjecting the oriented film to rubbing treatment. However, in this case, since the rubbing direction at the manufacture is limited, it is difficult to control precisely the refractive index anisotropy. In particular, there is involved a problem that it is especially difficult to obtain an optically compensatory film in which refractive index principal values in three directions of an optically anisotropic layer differ from each other.

In contrast, in recent years, a technology in which refractive index principal values in three directions of an optically anisotropic layer (device) are controlled by irradiating the optically anisotropic layer (device) with light is disclosed. For example, JP-A-2002-6138 discloses a method for obtaining an optically anisotropic layer in which when principal refractive indices in respective axis directions of X, Y and Z against a coordinate system consisting of the X axis and the Y axis in parallel to a film plane and the Z axis in a normal direction of the film plane are defined as nx, ny and nz, respectively, the principal refractive indices have the relationship of nx>ny>nz by orienting an optically uniaxial discotic liquid crystalline compound and then irradiating it with polarized light, or by cholesterically orienting an optically uniaxial rod-shaped liquid crystalline compound and then irradiating it with polarized light. Also, JP-A-2005-120091 discloses a method for obtaining an optically anisotropic layer in which principal refractive indices of an optically anisotropic layer have the relationship of nx>ny>nz by irradiating a polymerizable liquid crystal material containing a polymerizable mesogenic or liquid crystalline compound having a substituted cinnamate group in a side position thereof with polarized light.

Furthermore, a method for obtaining an optically anisotropic layer by applying three-dimensional molecular orientation control by means of photocrosslinking reaction is reported and disclosed. For example, EKISHO (Liquid Crystal), Vol. 7, No. 4, page 332 (2003), Macromol. Chem. Phys., Vol. 202, page 3087 (2001), and JP-A-11-189665 describe that in a high molecular liquid crystal containing a photoreactive group via a spacer composed of an alkylene group in a side chain of a high molecular compound, molecules are arranged in a parallel direction to the polarization direction as illustrated in FIG. 1. By following such a molecular arrangement, it is possible to obtain an optical characteristic represented by a refractive index ellipsoid in which values of ny and nz are substantially equal to each other and nx is larger than ny by irradiating linear polarized light from a vertical direction; and when a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of ny and nz are substantially equal to each other and nx is larger than ny is supposed, it is possible to obtain an optical characteristic represented by a refractive index ellipsoid obtained by rotating the subject refractive index ellipsoid around the X-axis as a rotation axis by an arbitrary rotation angle θ by irradiating p-polarized light from an oblique direction.

Moreover, JP-A-2003-307618 discloses a retardation film having an optical characteristic of a biaxial refractive index ellipsoid with principal refractive indices nx′, ny′ and nz′ in a direction by rotating a biaxial refractive index ellipsoid which has mutually different principal refractive indices nx, ny and nz in the X-axis, Y-axis and Z-axis directions, around the X-axis as a rotation axis by θ1 and around the Y-axis as a rotation axis by θ2 by continuously irradiating two linear polarized lights from an oblique direction and a method for producing the same. Also, JP-A-2003-307619 discloses a retardation film having an optical characteristic in conjunction with both a first refractive index ellipsoid having principal refractive indices nx, ny and nz in X-axis, Y-axis and Z-axis directions, respectively when a plane formed by the X-axis and the Y-axis is defined as a film plane inside and the Z-axis is defined as a thickness direction (with the relationship of the principal refractive indices of the first refractive index ellipsoid of nx>ny≧nz) and a second refractive index ellipsoid having principal refractive indices nx′, ny′ and nz′ in a direction by rotating the first refractive index ellipsoid around the Y-axis as a rotation axis by an angle θ1° and further rotating it around the Z-axis as a rotation axis by an angle θ2° (with the relationship of the principal refractive indices of the second refractive index ellipsoid of nx′>ny′≧nz′), and obtained by irradiating ultraviolet rays composed of a complete polarization component and a non-polarization component from a vertical direction to a horizontal plane while rotating an electric field vibration direction of the complete polarization component by a certain angle to an inclined axis of a glass substrate and subsequently turning the substrate over, followed by performing the irradiation in the same manner and a method for producing the same.

### SUMMARY OF THE INVENTION

However, there are optical characteristics which cannot be obtained even by employing the foregoing methods. In particular, it was very difficult to obtain (1) an optical characteristic represented by a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of nx and nz are substantially equal to each other and ny is smaller than nx; (2) an optical characteristic represented by a refractive index ellipsoid obtained by, when a refractive index ellipsoid in which values of nx and nz are substantially equal to each other and ny is smaller than nx is supposed, rotating the subject refractive index ellipsoid around the X-axis as a rotation axis by an arbitrary rotation angle θ; (3) an optical characteristic represented by a refractive index ellipsoid having the relationship of nx>nz>ny, namely an optical characteristic of 0<(nx−nz)/(nx−ny)<1; or (4) an optical characteristic represented by a refractive index ellipsoid obtained by, when a refractive index ellipsoid having the relationship of nx>nz>ny is supposed, rotating the subject refractive index ellipsoid around the foregoing X-axis as a rotation axis by an arbitrary angle θ.

Then, the invention is to provide an optically anisotropic film exhibiting an optical characteristic which is in general difficultly prepared, in particular an optically anisotropic film exhibiting (1) an optical characteristic represented by a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of nx and nz are substantially equal to each other and ny is smaller than nx, (2) an optical characteristic represented by a refractive index ellipsoid obtained by, when a refractive index ellipsoid in which values of nx and nz are substantially equal to each other and ny is smaller than nx is supposed, rotating the subject refractive index ellipsoid around the X-axis as a rotation axis by an arbitrary rotation angle θ, (3) an optical characteristic represented by a refractive index ellipsoid of nx>nz>ny, namely an optical characteristic of 0<(nx−nz)/(nx−ny<1), or (4) an optical characteristic represented by a refractive index ellipsoid obtained by, when a refractive index ellipsoid of nx>nz>ny is supposed, rotating the subject refractive index ellipsoid around the foregoing X-axis as a rotation axis by an arbitrary angle θ; an optically compensatory film using the optically anisotropic film and a method for producing the same.

In order to solve the foregoing problems, the present inventors made extensive and intensive investigations. As a result, it has been found that the problems of the invention can be solved by preparing an optically anisotropic film by using a composition containing a high molecular compound having a specified structure.
(1) A polymerizable monomer represented by the following Formula (I):

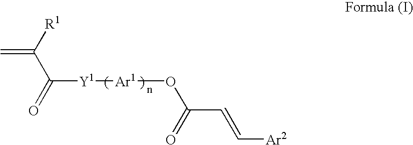

wherein R¹ represents a hydrogen atom or a substituted group; Y¹ represents an oxygen atom or —NR³—, wherein R³ represents a hydrogen atom or an alkyl group; Ar¹ and Ar² each independently represents an aromatic ring having from 1 to 10 carbon atoms, and each of Ar¹ and Ar²may have a substituted group; and n represents an integer of from 1 to 3.
(2) The polymerizable monomer according to (1), wherein Ar¹ is an aromatic ring represented by the following Formula (II):

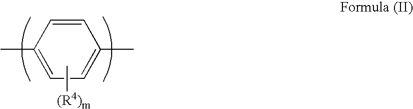

wherein R⁴represents a substituted group; m represents an integer of from 0 to 4; and when m is an integer of from 2 to 4, plural R⁴s may be the same or different.
(3) The polymerizable monomer according to (1), wherein Ar² is an optionally substituted benzene ring, furan ring or naphthalene ring.
(4) A high molecular compound formed of the polymerizable monomer according to (1).
(5) The high molecular compound according to (4), formed by using further a polymerizable monomer represented by the following Formula (III):

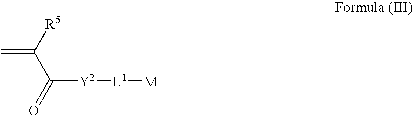

wherein R⁵ represents a hydrogen atom or a substituted group; L¹ represents a single bond or a divalent linking group; M represents a mesogen; and Y² represents —NR⁶—, wherein R⁶ represents an alkyl group or a hydrogen atom, or —O—.
(6) An optically anisotropic film formed of a composition containing the high molecular compound according to (4).
(7) An optically compensatory film comprising a substrate having thereon the optically anisotropic film according to (6).
(8) The optically compensatory film according to (7), wherein the substrate comprises a cellulose-based polymer or a cycloolefin-based polymer.
(9) A polarizing plate comprising two protective films having a polarizer interposed therebetween, wherein at least one of the two protective films is the optically anisotropic film according to (6) or the optically compensatory film according to (7).
(10) A liquid crystal display device comprising the optically anisotropic film according to (6) or the optically compensatory film according to (7) or (8).
(11) A method for producing the optically compensatory film according to (7), comprising (a) coating a composition containing the high molecular compound according to (4) or (5) on a substrate; and (b) irradiating it with polarized light from a single direction to the substrate.
(12) The method for producing the optically compensatory film according to (11), wherein the polarized light is irradiated from a vertical direction to the substrate.
(13) The method for producing the optically compensatory film according to (11), wherein in the (b), the polarized light is irradiated from an oblique direction to the substrate.
(14) The method for producing the optically compensatory film according to (11), wherein the polarized light to be used for the irradiation with polarized light is p-polarized light or s-polarized light.
(15) The method for producing the optically compensatory film according to (11), wherein the (b) is carried out at a temperature of not higher than a glass transition temperature of the high molecular compound.
(16) The method for producing the optically compensatory film according to (11), further comprising (c) thermally treating the substrate and/or the composition after the (b).

### BRIEF DESCRIPTION OF THE DRAWINGS

FIG. 1 is a conceptual view to exhibit an optical characteristic of an optically anisotropic film obtainable upon irradiation of a related-art photoreactive group-containing high molecular compound with polarized light.

FIG. 2 is a schematic view when a photoreactive group-containing high molecular compound of the invention is irradiated with polarized light from a vertical direction to a substrate.

FIG. 3 is a schematic view when a photoreactive group-containing high molecular compound of the invention is irradiated with p-polarized light from an oblique direction to a substrate.

FIG. 4 is an explanatory view of a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of nx and nz are substantially equal to each other and ny is smaller than nx.

FIG. 5 is an explanatory view of a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, nx, ny and nz have the relationship of nx>nz>ny.

FIG. 6 is a schematic view seen from a Y-axis direction when a photoreactive group-containing high molecular compound of the invention is irradiated with s-polarized light from an oblique direction to a substrate.

FIG. 7 is a schematic view seen from an X-axis direction when a photoreactive group-containing high molecular compound of the invention is irradiated with s-polarized light from an oblique direction to a substrate.

FIG. 8 is an explanatory view of a refractive index ellipsoid obtained by, when in the case where principal refractive indices in respective axis directions of X, Y and Z against a coordinate system consisting of the X axis and the Y axis in parallel to a film plane and the Z axis in a normal direction of the film plane are defined as nx, ny and nz, respectively, a refractive index ellipsoid in which values of nx and nz are substantially equal to each other and ny is smaller than nx is supposed, rotating the refractive index ellipsoid around the X-axis as a rotation axis by an arbitrary rotation angle θ.

FIG. 9 is an explanatory view of a refractive index ellipsoid obtained by, when in the case where principal refractive indices in respective axis directions of X, Y and Z against a coordinate system consisting of the X axis and the Y axis in parallel to a film plane and the Z axis in a normal direction of the film plane are defined as nx, ny and nz, respectively, a refractive index ellipsoid having the relationship of nx>nz>ny is supposed, rotating the refractive index ellipsoid around the X-axis as a rotation axis by an arbitrary rotation angle θ.

### DESCRIPTION OF SPECIFIC EMBODIMENTS

The contents of the invention are hereunder described in detail. In this description, the numerical range expressed by the wording “a number to another number” means the range that falls within the range from the former number indicating the lowermost limit of the range and the latter number indicating the uppermost limit thereof.

In the invention, a direction where an in-plane refractive index of a film becomes maximal is defined as “X-axis”; a vertical direction to the X-axis is defined as “Y-axis”; a thickness direction of the film is defined as “Z-axis”; and refractive indices in the respective axis directions are defined as “nx”, “ny” and “nz”, respectively.

Also, the state that nx and nz are substantially equal to each other means, for example, a state having an equal optical characteristic to that of an optically anisotropic layer composed of a vertically oriented discotic liquid crystal layer.

In the description, Re(λ) and Rth(λ) each indicate the in-plane retardation and the thickness direction retardation of the film at a wavelength λ. Re(λ) is measured by applying a light having a wavelength of λ nm in the normal direction of the film, using KOBRA-21ADH or WR (by Oji Scientific Instruments).

When the film tested is represented by an uniaxial or biaxial refractive index ellipsoid, then its Rth(λ) is computed according to the method mentioned below.

With the in-plane slow axis (judged by KOBRA 21ADH or WR) taken as the inclination axis (rotation axis) of the film (in case where the film has no slow axis, the rotation axis of the film may be in any in-plane direction of the film), Re(λ) of the film is measured at 6 points in all thereof, from −50° to +50° relative to the normal direction of the film at intervals of 10°, by applying a light having a wavelength of λ nm from the inclined direction of the film. Based on the thus-determined retardation data of Re(λ), the mean refractive index and the inputted film thickness, Rth(λ) of the film is computed with KOBRA 21ADH or WR.

With the in-plane slow axis from the normal direction taken as the rotation axis thereof, when the film has a zero retardation value at a certain inclination angle, then the symbol of the retardation value of the film at an inclination angle larger than that inclination angle is changed to a negative one, and then applied to KOBRA 21ADH or WR for computation.

With the slow axis taken as the inclination axis (rotation axis) (in case where the film has no slow axis, the rotation axis of the film may be in any in-plane direction of the film), the retardation values of the film are measured in any inclined two directions; and based on the data and the mean refractive index and the inputted film thickness, Rth may be computed according to the following formulae (1) and (2):

$$\begin{matrix}Re ( θ ) = [ n x - \frac{n y \times n z}{\sqrt{\begin{matrix}{\{ n y sin ( {sin}^{- 1} ( \frac{sin ( - θ )}{n x} ) ) \}}^{2} + \\ {\{ n z cos ( {sin}^{- 1} ( \frac{sin ( - θ )}{n x} ) ) \}}^{2}\end{matrix}}} ] \times \\ \frac{d}{cos \{ {sin}^{- 1} ( \frac{sin ( - θ )}{n x} ) \}}\end{matrix} \qquad ( 1 )$$

wherein Re(θ) means the retardation value of the film in the direction inclined by an angle θ from the normal direction; nx means the in-plane refractive index of the film in the slow axis direction; ny means the in-plane refractive index of the film in the direction vertical to nx; nz means the refractive index of the film vertical to nx and ny.

Rth=((nx+ny)/2−nz)×d. (2)

When the film to be tested could not be represented by a monoaxial or biaxial index ellipsoid, or that is, when the film does not have an optical axis, then its Rth(λ) may be computed according to the method mentioned below.

With the in-plane slow axis (judged by KOBRA 21ADH or WR) taken as the inclination axis (rotation axis) of the film, Re(λ) of the film is measured at 11 points in all thereof, from −50° to +50° relative to the normal direction of the film at intervals of 10°, by applying a light having a wavelength of λ nm from the inclined direction of the film. Based on the thus-determined retardation data of Re(λ), the mean refractive index and the inputted film thickness, Rth(λ) of the film is computed with KOBRA 21ADH or WR.

The mean refractive index may be used values described in catalogs for various types of optical films. When the mean refractive index has not known, it may be measured with Abbe refractometer. The mean refractive index for major optical film is described below: cellulose acetate (1.48), cycloolefin polymer (1.52), polycarbonate (1.59), polymethylmethacrylate (1.49), polystyrene (1.59). The mean refractive index and the film thickness are inputted in KOBRA 21ADH or WR, nx, ny and nz are computed therewith. From the thus-computed data of nx, ny and nz, Nz=(nx−nz)/(nx−ny) is further computed.

In this specification, the terms “parallel” and “orthogonal” mean that the angle falls within the range of a strict angle and less than ±10°. This range is preferably less than ±5°, and more preferably less than ±2° in terms of an error from the strict angle. Also, the “slow axis” means a direction where a refractive index becomes maximal. A measurement wavelength of the refractive index is a value at λ=550 nm in a visible light region unless otherwise indicated specifically.

A material to be used for the preparation and a production method and the like of each of a polymerizable monomer, a high molecular compound, an optically anisotropic film and an optically compensatory film of the invention are hereunder described in detail.

A polymerizable monomer of the invention is characterized by being represented by the following Formula (I).

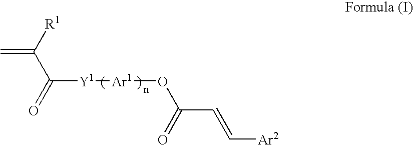

In the formula, R¹ represents a hydrogen atom or a substituted group; and when R¹ represents a substituted group, preferred examples thereof include an alkyl group and a halogen group. R¹ is more preferably a hydrogen atom, an alkyl group having from 1 to 6 carbon atoms, or a chloro group, and further preferably a hydrogen atom, a methyl group, an ethyl group, or a chloro group.

Y¹ represents an oxygen atom or —NR³— (wherein R³ represents a hydrogen atom or an alkyl group (preferably an alkyl group having from 1 to 6 carbon atoms)), preferably an oxygen atom or —NH—, and more preferably an oxygen atom.

Ar¹ and Ar² each independently represents an aromatic ring having from 1 to 10 carbon atoms, each of Ar¹ and Ar² may have a substituted group. Ar¹ is preferably a benzene ring, a thiophene ring, a furan ring, or a naphthalene ring, and more preferably a benzene ring. Of the benzene rings, one represented by the following Formula (II) is preferable.

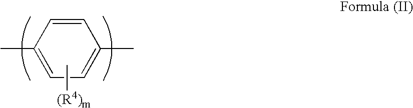

In the formula, R⁴ represents a substituted group; and m represents an integer of from 0 to 4. When m is an integer of from 2 to 4, plural R⁴s may be the same or different. m is preferably an integer of from 0 to 2.

When Ar¹ has a substituted group, preferred examples of the substituted group represented by R⁴ include an alkyl group having from 1 to 6 carbon atoms, an alkoxyl group having from 1 to 6 carbon atoms, an alkylthio group having from 1 to 6 carbon atoms, an alkylcarbonyl group having from 1 to 6 carbon atoms, an alkylcarbonyloxy group having from 1 to 6 carbon atoms, an alkylcarbonylthio group having from 1 to 6 carbon atoms, an alkylcarbonylamino group having from 1 to 6 carbon atoms, a halogen group, a cyano group, and a nitro group; and more preferred examples of the substituted group include a methyl group, an ethyl group, a methoxy group, a fluoro group, a chloro group, a bromo group, and a cyano group.

These substituted groups may be substituted with other substituted group; and in this case, preferred examples of the substituted group are synonymous with those as described above. When two or more substituted groups are present, the respective substituted groups may be the same or different. If possible, the substituted groups may be taken together to form a ring.

Ar² is preferably a benzene ring, a furan ring, or a naphthalene ring, and more preferably a benzene ring. When Ar² has a substituted group, examples of the substituted group include an alkyl group, an alkenyl group, an alkynyl group, an alkoxyl group, an alkylthio group, an alkylcarbonyl group, an alkylcarbonyloxy group, an alkylcarbonylthio group, an alkylcarbonylamino group, an alkyloxycarbonyloxy group, an alkyloxycarbonylthio group, alkyloxycarbonylamino group, an alkylthiocarbonyloxy group, an alkylaminocarbonylamino group, an alkylaminocarbonyloxy group, a halogen group, a cyano group, a nitro group, and an aryl group; and more preferred examples of the substituted group include an alkyl group having from 1 to 20 carbon atoms, an alkoxyl group having from 1 to 20 carbon atoms, an alkylthio group having from 1 to 20 carbon atoms, an alkylcarbonyl group having from 1 to 20 carbon atoms, an alkylcarbonyloxy group having from 1 to 20 carbon atoms, an alkylcarbonylamino group having from 1 to 20 carbon atoms, an alkyloxycarbonyloxy group having from 1 to 20 carbon atoms, an alkyloxycarbonylthio group having from 1 to 20 carbon atoms, an alkyloxycarbonylamino group having from 1 to 20 carbon atoms, an alkylaminocarbonyloxy group having from 1 to 20 carbon atom, a fluoro group, a chloro group, a bromo group, a cyano group, and a nitro group.

These substituted groups may be substituted with other substituted group; and in this case, preferred examples of the substituted group are synonymous with those as described above. When two or more substituted groups are present, the respective substituted groups may be the same or different. If possible, the substituted groups may be taken together to form a ring.

n represents an integer of from 1 to 3, and preferably 1 or 2.

Specific examples of the polymerizable monomer represented by the Formula (I) of the invention are given below, but it should not be construed that the invention is limited to these specific examples.

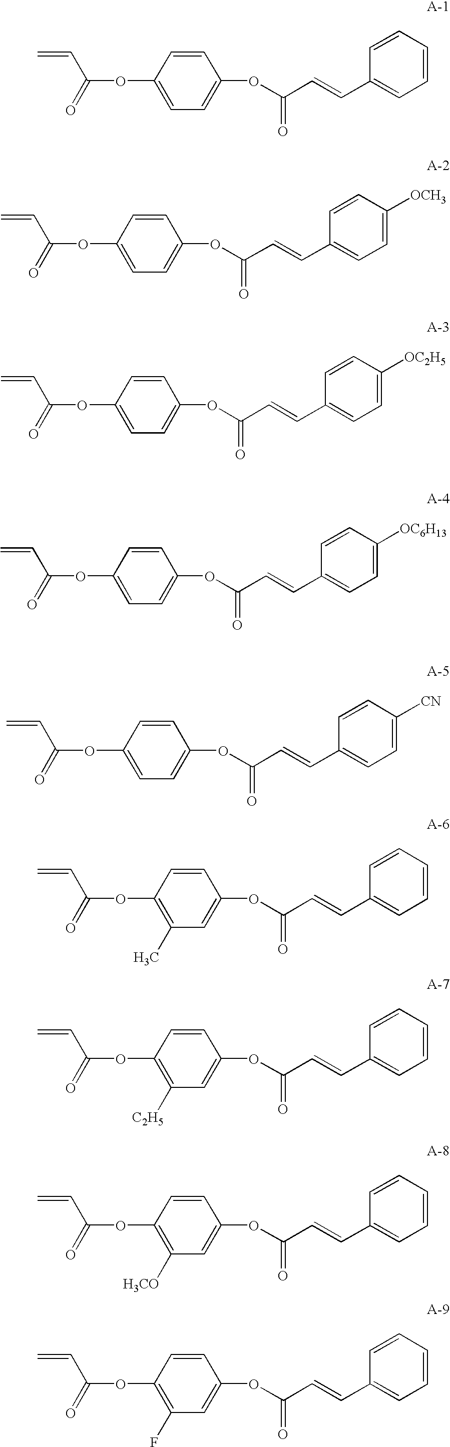

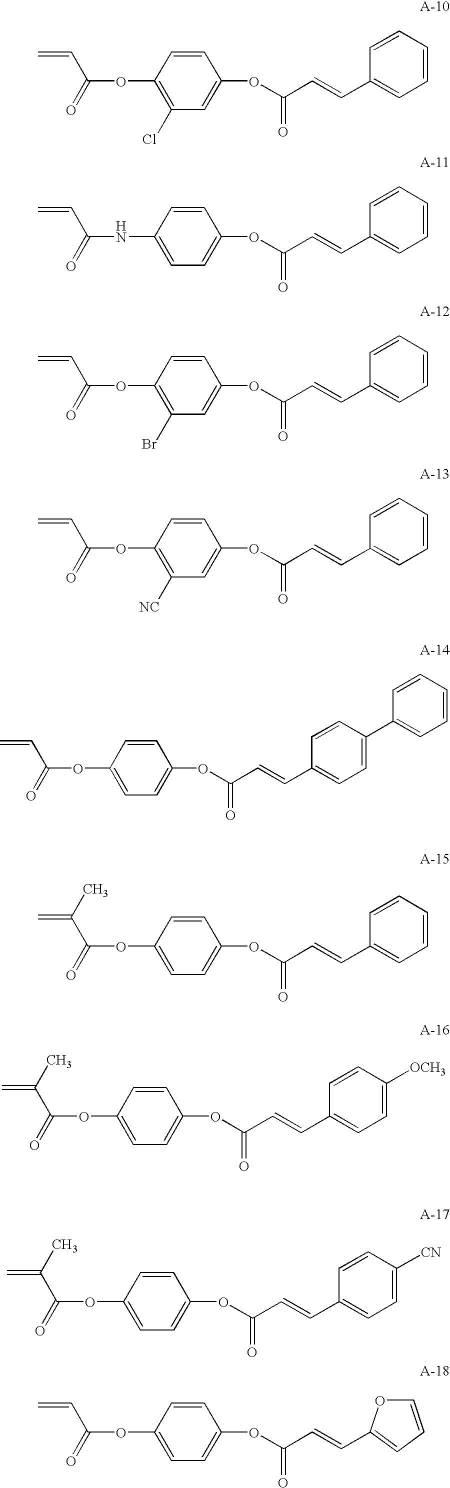

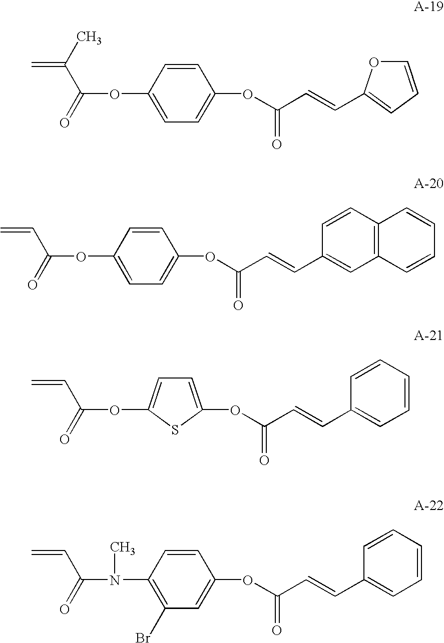

The compound represented by the Formula (I) which is the polymerizable monomer of the invention can be synthesized by employing an already-known synthesis method. For example, a method represented by the following Synthesis Scheme 1 can be enumerated.

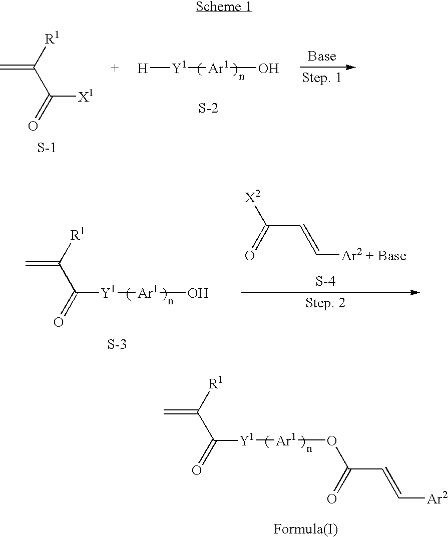

In the foregoing Scheme 1, X¹ and X² each represents a split-off group. Preferred examples of the split-off group include a halogen atom, a mesyl group, and a tosyl group. Besides, R¹, Y¹, Ar¹, Ar² and n are synonymous with those as defined in the foregoing Formula (I).

In the Step 1, a compound represented by the formula S-2 is dissolved in a solvent and allowed to react with a compound represented by the formula S-1 in the presence of a base, thereby synthesizing an intermediate represented by the formula S-3. As the solvent, ether based solvents such as tetrahydrofuran, amide based solvents such as dimethylacetamide, and halogen based solvents such as dichloromethane are suitable. As the base, any of inorganic and organic bases may be employed, and organic bases such as triethylamine and diisopropylethylamine and inorganic bases such as potassium carbonate and potassium hydrogencarbonate are suitable. An amount of the base which is used is preferably in the range of from 0.5 to 10 equivalents, and more preferably in the range of from 0.7 to 2 equivalents, relative to S-1. A reaction temperature is usually from −10° C. to a boiling point of the solvent, and preferably from 0° C. to room temperature. A reaction time is usually from 10 minutes to one day, and preferably from one hour to 12 hours.

In the Step 2, the thus-obtained compound represented by the formula S-3 is dissolved in a solvent and allowed to react with a compound represented by the formula S-4 in the presence of a base, whereby a desired monomer represented by the Formula (I) of the invention can be synthesized. As the solvent, ether based solvents such as tetrahydrofuran, amide based solvents such as dimethylacetamide, and halogen based solvents such as dichloromethane are suitable. As the base, any of inorganic and organic bases may be employed, and organic bases such as triethylamine and diisopropylethylamine and inorganic bases such as potassium carbonate and potassium hydrogencarbonate are suitable. An amount of the base which is used is preferably in the range of from 0.5 to 10 equivalents, and more preferably in the range of from 0.7 to 2 equivalents, relative to S-3. A reaction temperature is usually from −10° C. to a boiling point of the solvent, and preferably from 0° C. to room temperature. A reaction time is usually from 10 minutes to one day, and preferably from one hour to 12 hours.

The high molecular compound of the invention can be formed by using the polymerizable monomer of the invention. The high molecular compound of the invention may be a polymer containing only one kind of the polymerizable monomer represented by the foregoing Formula (I) or may be a polymer containing two or more kinds of the polymerizable monomer represented by the foregoing Formula (I). The high molecular compound of the invention may also contain one or two or more kinds of repeating units other than the foregoing respective repeating units. The repeating units other than the foregoing respective repeating units are not particularly limited so far as the gist of the invention is not deviated. Preferred examples of such a repeating unit include repeating units derived from usual radical polymerizable monomers.

It is preferable that the high molecular compound of the invention contains a repeating unit derived from at least one mesogen group-containing monomer. As such a mesogen group-containing monomer, a monomer represented by the following Formula (III) is preferable.

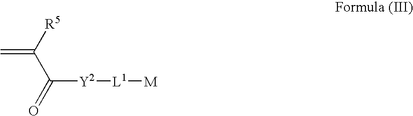

In the foregoing Formula (III), R⁵ represents a hydrogen atom or a substituted group; examples of the substituted group include substituted groups selected from the group of substituted groups as enumerated for R¹ of the foregoing Formula (I); and preferred examples thereof can also be thought to be the same.

In the foregoing Formula (III), L¹ represents a single bond or a divalent linking group. When L¹ is a divalent linking group, a divalent linking group selected from the group consisting of an alkylene group, an alkenylene group, an arylene group, a divalent heterocyclic residue group, —CO—, —NR⁷— (wherein R⁷ represents an alkyl group having from 1 to 6 carbon atoms or a hydrogen atom), —O—, —S—, —SO—, —SO₂—, and a combination thereof is preferable. The alkylene group preferably has from 1 to 12 carbon atoms. The alkenylene group preferably has from 2 to 12 carbon atoms. The arylene group preferably has from 6 to 10 carbon atoms. If possible, each of the alkylene group, the alkenylene group and the arylene group may be substituted with a substituted group (for example, an alkyl group, a halogen atom, a cyano group, an alkoxy group, and an acyloxy group).

L¹ is preferably a single bond or contains —O—, —CO—, —NR⁷— (wherein R⁷ represents an alkyl group having from 1 to 6 carbon atoms or a hydrogen atom), an alkylene group, or an arylene group; and preferably L¹ is more a single bond or contains —O—, an alkylene group, or an arylene group.

Specific examples of the structure of L¹ are given below, but it should not be construed that the invention is limited to these specific examples. Combinations of two or more of the following specific examples are also preferable.

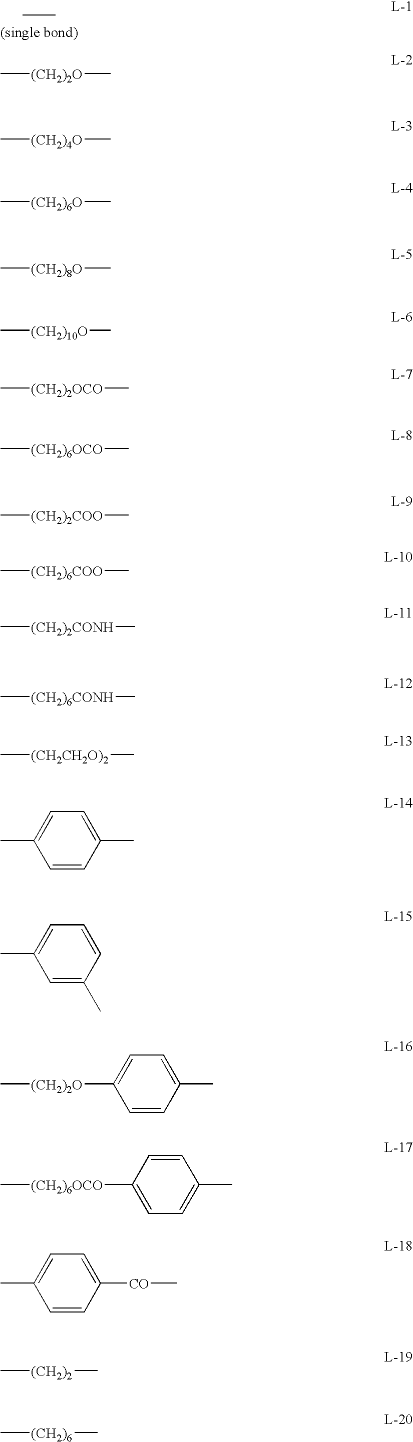

Y² represents —NR⁶— or —O—, and preferably —NH— or —O—. Here, R⁶ represents an alkyl group (preferably an alkyl group having from 1 to 6 carbon atoms) or a hydrogen atom.

In the Formula (III), M represents a mesogen, and structures described in Makromol. Chem., Vol. 190, page 2255 (1989) and Advanced Materials, Vol. 5, page 107 (1993) can be used. A mesogen group represented by the following Formula (IV) is more preferable.

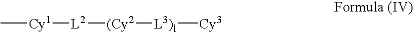

In the foregoing Formula (IV), L² and L³ each independently represents a single bond or a divalent linking group; Cy¹, Cy² and Cy³ each independently represents a divalent cyclic group; and 1 represents an integer of from 0 to 2. When 1 is 2, two L³s may be the same or different; and two Cy²s may be the same or different.

In the Formula (IV), it is preferable that L² and L³ are each independently a divalent linking group selected from the group consisting of —O—, —S—, —CO—, —NR⁸—, a divalent chain group, a divalent cyclic group, and a combination thereof or a single bond. R⁸represents an alkyl group having from 1 to 7 carbon atoms or a hydrogen atom; preferably an alkyl group having from 1 to 4 carbon atoms or a hydrogen atom; more preferably a methyl group, an ethyl group, or a hydrogen atom; and further preferably a hydrogen atom.

The divalent chain group is preferably an alkylene group, an alkenylene group, or an alkynylene group, each of which may have a substituted group. As the substituted group, a halogen atom is preferable. Of the divalent chain groups, an alkylene group and an alkenylene group are preferable; and an unsubstituted alkylene group and an unsubstituted alkenylene group are more preferable. The alkylene group may be branched. The alkylene group preferably has from 1 to 12 carbon atoms, more preferably from 2 to 10 carbon atoms, and further preferably from 2 to 8 carbon atoms. The alkenylene group may be branched. The alkenylene group preferably has from 2 to 12 carbon atoms, more preferably from 2 to 10 carbon atoms, and further preferably from 2 to 8 carbon atoms.

The alkynylene group maybe branched. The alkynylene group preferably has from 2 to 12 carbon atoms, more preferably from 2 to 10 carbon atoms, and further preferably from 2 to 8 carbon atoms.

Specific examples of the divalent chain group include an ethylene group, a trimethylene group, a propylene group, a tetramethylene group, a 2-methyl-tetramethylene group, a pentamethylene group, a hexamethylene group, an octamethylene group, a 2-butenylene group, and a 2-butynylene group.

The divalent cyclic group is synonymous with Cy¹, Cy² and Cy³ as described later, and preferred examples thereof are also the same.

In the Formula (IV), 1 is preferably 0 or 1.

In the Formula (IV), Cy¹, Cy² and Cy³ each independently represents a divalent cyclic group. A ring which is contained in the cyclic group is preferably a 5-membered ring, a 6-membered ring, or a 7-membered ring, more preferably a 5-membered ring or a 6-membered ring, and further preferably a 6-membered ring. A ring which is contained in the cyclic group may be a monocycle or a fused ring and is preferably a monocycle. The ring which is contained in the cyclic group may be any of an aromatic ring, an aliphatic ring and a hetero ring. Examples of the aromatic ring include a benzene ring and a naphthalene ring. Examples of the aliphatic ring include a cyclohexane ring. Examples of the hetero ring include a pyridine ring and a pyrimidine ring. As a benzene ring-containing cyclic group, a 1,4-phenylene group is preferable. As a naphthalene ring-containing cyclic group, a naphthalene-1,5-diyl group and a naphthalene-2,6-diyl group are preferable. As a cyclohexane ring-containing cyclic group, a 1,4-cyclohexylene group is preferable. As a pyridine ring-containing cyclic group, a pyridine-2,5-diyl group is preferable. As a pyrimidine ring-containing cyclic group, a pyrimidine-2,5-diyl group is preferable.

The cyclic group may have a substituted group. Examples of the substituted group include a halogen atom, a cyano group, a nitro group, an alkyl group having from 1 to 5 carbon atoms, a halogen atom-substituted alkyl group having from 1 to 5 carbon atoms, an alkoxy group having from 1 to 5 carbon atoms, an alkylthio group having from 1 to 5 carbon atoms, an acyloxy group having from 2 to 6 carbon atoms, an alkoxycarbonyl group having from 2 to 6 carbon atoms, a carbamoyl group, a carbamoyl group substituted with an alkyl group having from 2 to 6 carbon atoms, and an acylamino group having from 2 to 6 carbon atoms.

Specific examples of the monomer constituting the photoreactive group-containing repeating unit represented by the Formula (III) are given below, but it should not be construed that the invention is limited to these specific examples.

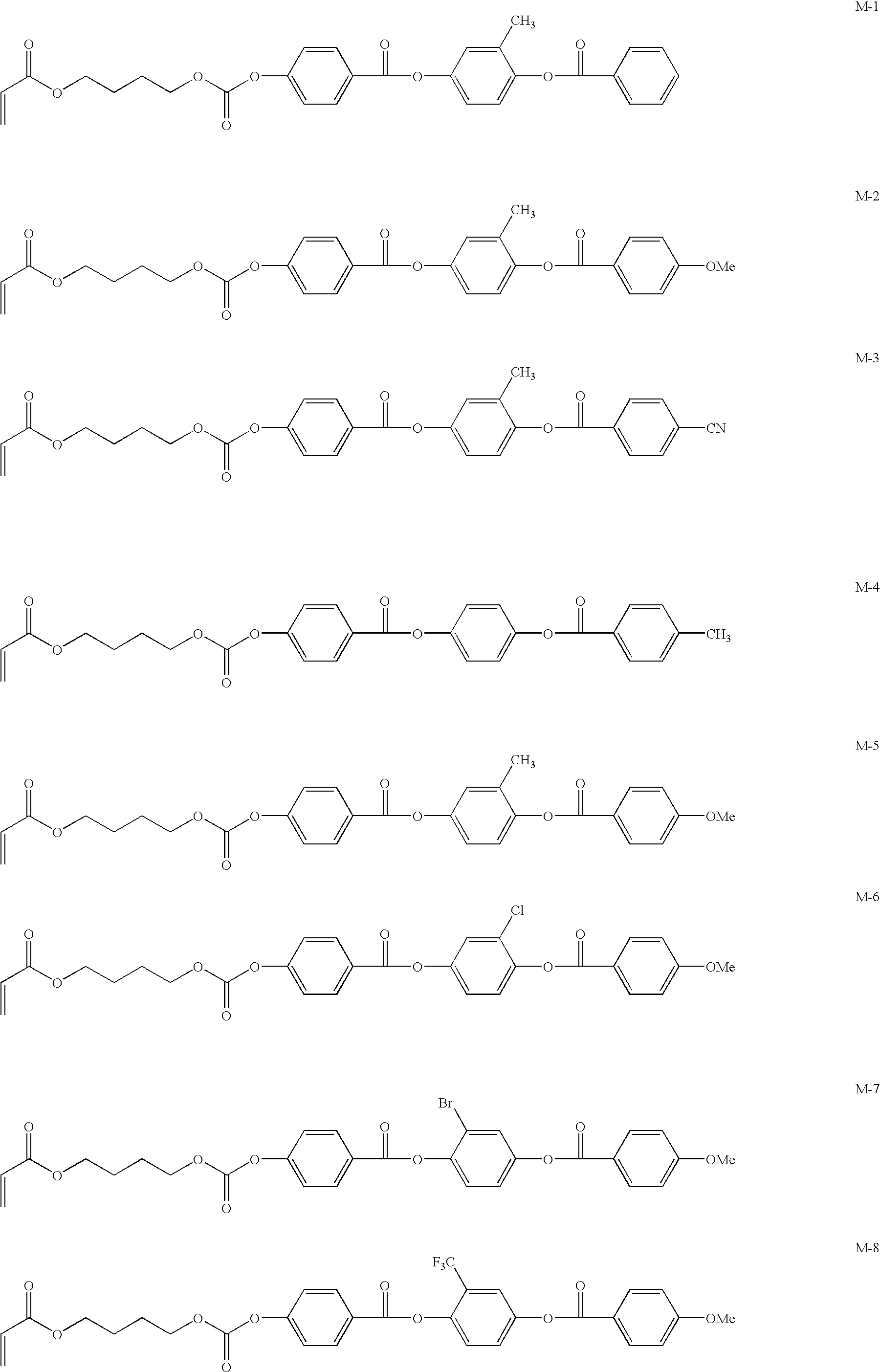

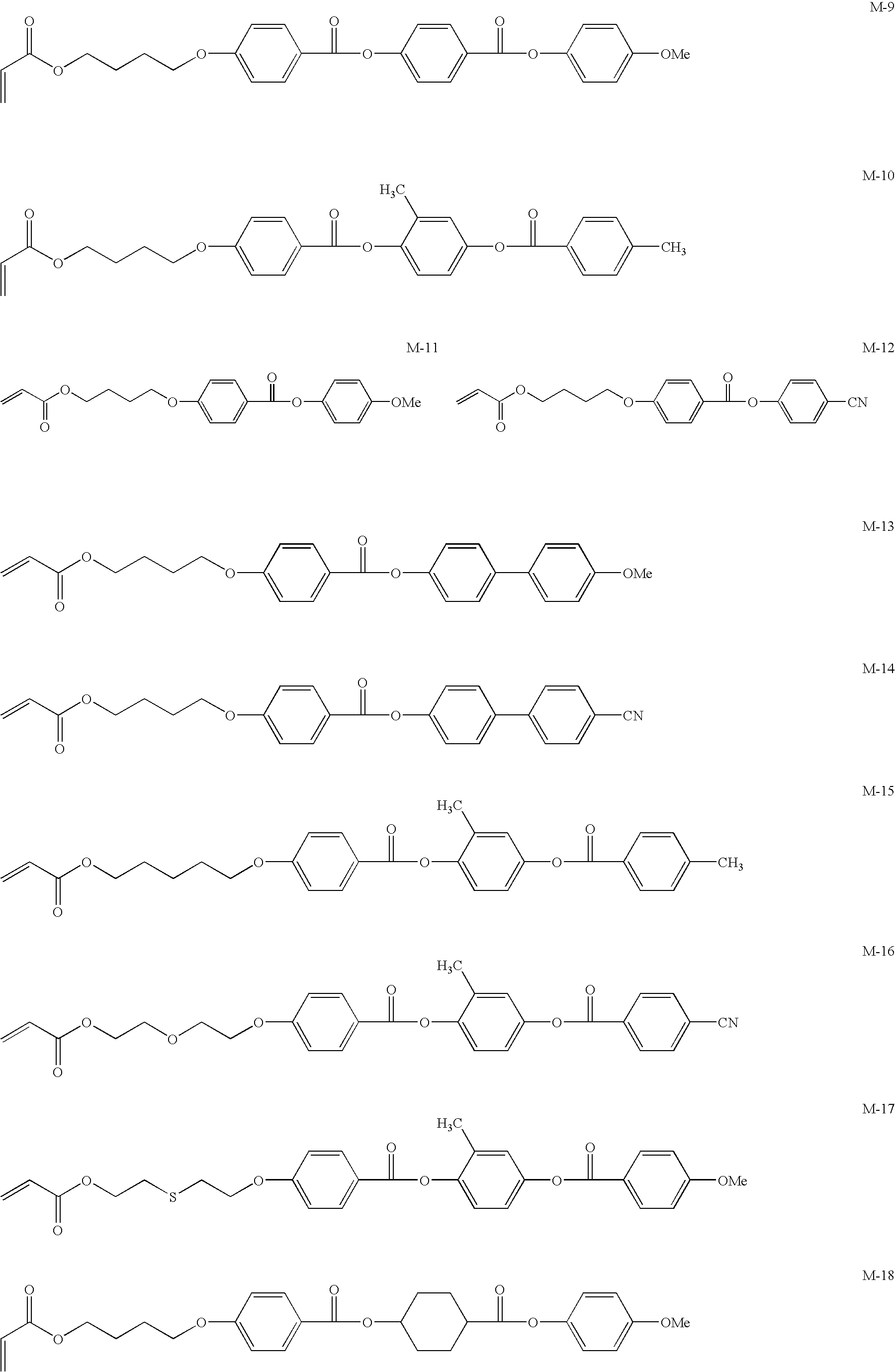

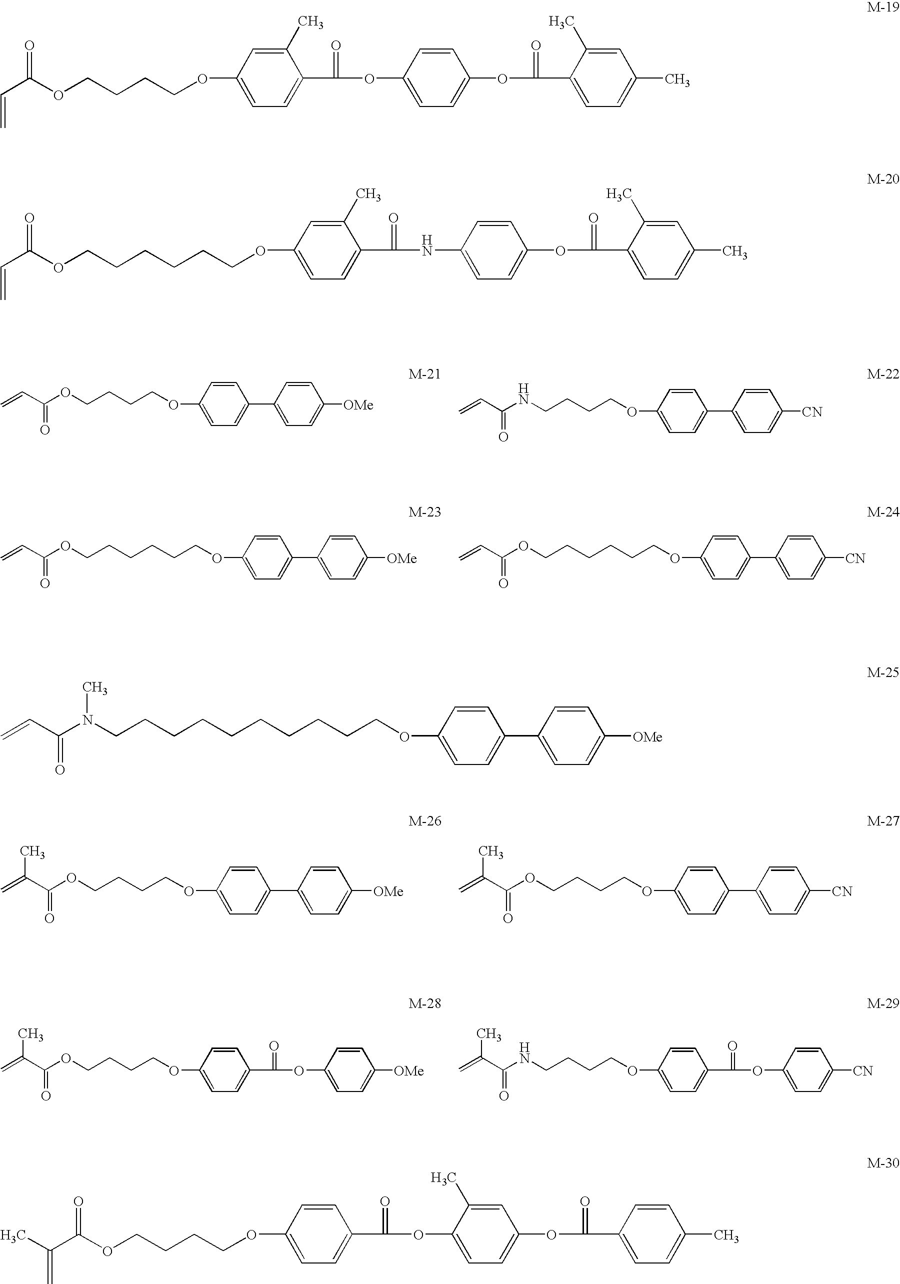

The high molecular compound of the invention is preferably a copolymer containing a repeating unit derived from at least one monomer represented by the foregoing Formula (I) and a repeating unit derived from at least one monomer represented by the foregoing Formula (III). Of the monomers constituting the subject polymer, an amount of the monomer represented by the foregoing Formula (I) is preferably not more than 30% by mole, more preferably not more than 20% by mole, and further preferably not more than 10% by mole relative to the total molar number of the monomers constituting the subject polymer. A lower limit value thereof is not particularly defined but is preferably 1% by mole or more.

Of the monomers constituting the subject polymer, an amount of the monomer represented by the foregoing Formula (III) is preferably from 50% by mole to 99% by mole, more preferably from 80% by mole to 99% by mole, and further preferably from 90% by mole to 99% by mole relative to the total molar number of the monomers constituting the subject polymer.

In the invention, the photoreactive group-containing high molecular compound may or may not have liquid crystallinity.

The high molecular compound of the invention can contain a generally known arbitrary repeating unit other than the repeating unit derived from the polymerizable monomer represented by the foregoing Formula (I) and the repeating unit derived from the polymerizable monomer represented by the foregoing Formula (III). The kind of the arbitrary repeating unit to be used and the molar percentage of the subject arbitrary repeating unit are properly chosen depending upon conditions such as the kind of the monomer to be used and desired physical properties. For example, the content of the subject arbitrary repeating unit is preferably from 30 to 1% by mole, more preferably from 20 to 1% by mole, and further preferably from 10 to 1% by mole relative to the total molar number of the monomers constituting the polymer.

A weight-average molecular weight of the high molecular compound of the invention is preferably from 1,000 to 1,000,000, more preferably from 1,000 to 500,000, and further preferably from 5,000 to 100,000. The weight-average molecular weight can be measured as a converted value into polystyrene (PS) using gel permeation chromatography (GPC).

The method for producing the high molecular compound of the invention is not particularly limited, and a polymerization method, for example, cationic polymerization or radical polymerization utilizing a vinyl group and anionic polymerization can be employed. Of these, radical polymerization is especially preferable because it can be used for various purposes. As a polymerization initiator of the radical polymerization, known compounds such as radical thermal polymerization initiators and radical photopolymerization initiators can be used. In particular, it is preferred to use a radical thermal polymerization initiator. Here, the radical thermal polymerization initiator is a compound capable of emitting a radical upon being heated at a decomposition temperature or higher. Examples of such a radical thermal polymerization initiator include diacyl peroxides (for example, acetyl peroxide and benzoyl peroxide), ketone peroxides (for example, methyl ethyl ketone peroxide and cyclohexanone peroxide), hydroperoxides (for example, hydrogen peroxide, tert-butyl hydroperoxide, and cumene hydroperoxide), dialkyl peroxides (for example, di-tert-butyl peroxide, dicumyl peroxide, and dilauroyl peroxide), peroxy esters (for example, tert-butyl peroxyacetate and tert-butyl peroxypivalate), azo based compounds (for example, azobisisobutyronitrile and azobisisovaleronirile), and persulfates (for example, ammonium persulfate, sodium persulfate, and potassium persulfate). These radical thermal polymerization initiators can be used singly or can be used in combination of two or more kinds thereof.

The foregoing radical polymerization method is not particularly limited, and known methods such as an emulsion polymerization method, a suspension polymerization method, a block polymerization method, and a solution polymerization method can be employed. The solution polymerization which is a typical radical polymerization method is more specifically described. Outlines of other polymerization methods are also similar, and details thereof are described in, for example, Kobunshi Kagaku Jikkenho (Polymer Science Experimental Method), compiled by the Society of Polymer Science, Japan (Tokyo Kagaku Dojin Co., Ltd., 1981).

For the purpose of achieving the foregoing solution polymerization, an organic solvent is used. Such an organic solvent can be arbitrarily chosen within the scope where the object and effects of the invention are not impaired. Such an organic solvent is usually an organic compound having a boiling point in the range of from 50 to 200° C. under atmospheric pressure, and an organic solvent capable of dissolving uniformly the respective constitutional components therein is desirable. Preferred examples of the organic solvent include alcohols such as isopropanol and butanol; ethers such as dibutyl ether, ethylene glycol dimethyl ether, tetrahydrofuran, and dioxane; ketones such as acetone, methyl ethyl ketone, methyl isobutyl ketone, and cyclohexanone; esters such as ethyl acetate, butyl acetate, amyl acetate, and γ-butyrolactone; amides such as dimethylamide and dimethylformamide; and aromatic hydrocarbons such as benzene, toluene, and xylene. These organic solvents can be used singly or in combination of two or more kinds thereof. A water-mixed organic solvent in which water is used jointly with the foregoing organic solvent can also be applied from the viewpoint of solubility of the monomer or the polymer to be formed.

A condition for the solution polymerization is not particularly limited, and for example, the solution polymerization is desirably carried out by heating at a temperature in the range of from 50 to 200° C. for from 10 minutes to 30 hours. In order that the emitted radical may not be inactivated, it is desirable that an inert gas purge is carried out not only during the solution polymerization but prior to initiation of the solution polymerization. In general, a nitrogen gas is suitably used as the inert gas.

In order to obtain the high molecular compound containing a repeating unit derived from at least one monomer represented by the Formula (I) within a desired molecular weight range, a radical polymerization method using a chain transfer agent is especially effective. As the chain transfer agent, any of mercaptans (for example, octyl mercaptan, decyl mercaptan, dodecyl mercaptan, tert-dodecyl mercaptan, octadecyl mercaptan, thiophenol, and p-nonylthiophenol), polyhalogenated alkyls (for example, carbon tetrachloride, chloroform, 1,1,1-trichloroethane, and 1,1,1-tribromooctane), and low-activity monomers (for example, α-methylstyrene and an α-methylstyrene dimer) can be used; and mercaptans having from 4 to 16 carbon atoms are preferable. An amount of the chain transfer agent to be used is remarkably influenced by activity of the chain transfer agent, a combination of the monomers, a polymerization condition, and the like and must be precisely controlled. The amount of the chain transfer agent to be used is preferably from 0.01% by mole to 50% by mole, more preferably from 0.05% by mole to 30% by mole, and further preferably from 0.08% by mole to 25% by mole relative to the total molar number of the monomers to be used. Such a chain transfer agent may be made present in the system simultaneously with the subjective monomer whose degree of polymerization must be controlled during the polymerization process, and an addition method thereof is not particularly limited. The chain transfer agent may be added by dissolving in the monomer. It is also possible to add the chain transfer agent separately from the monomer.

Specific examples of the photoreactive group-containing high molecular compound which is used in the invention are given below, but it should not be construed that the compound which is useful in the invention is limited thereto. Numerals in the following formulae mean a weight percentage of the respective monomer components. Also, Mw represents a weight-average molecular weight.

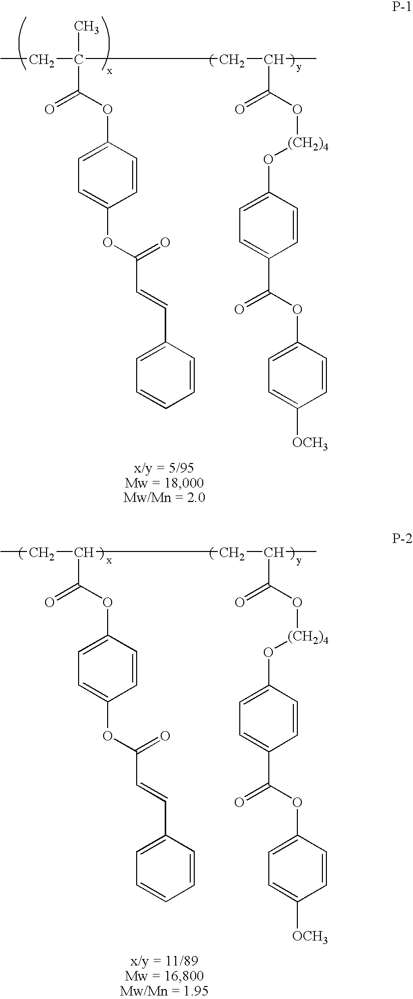

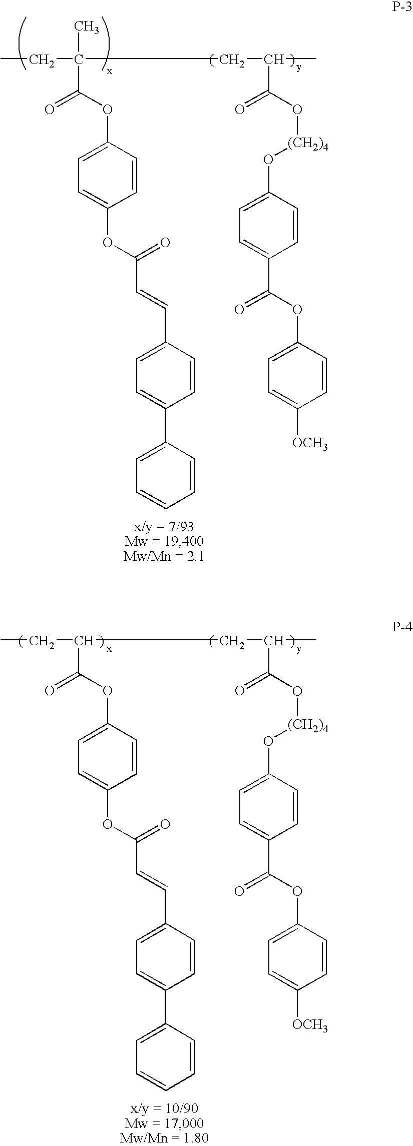

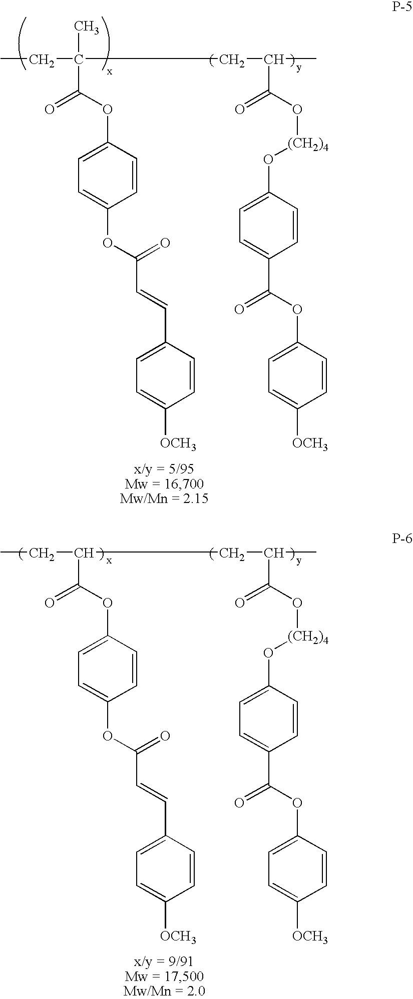

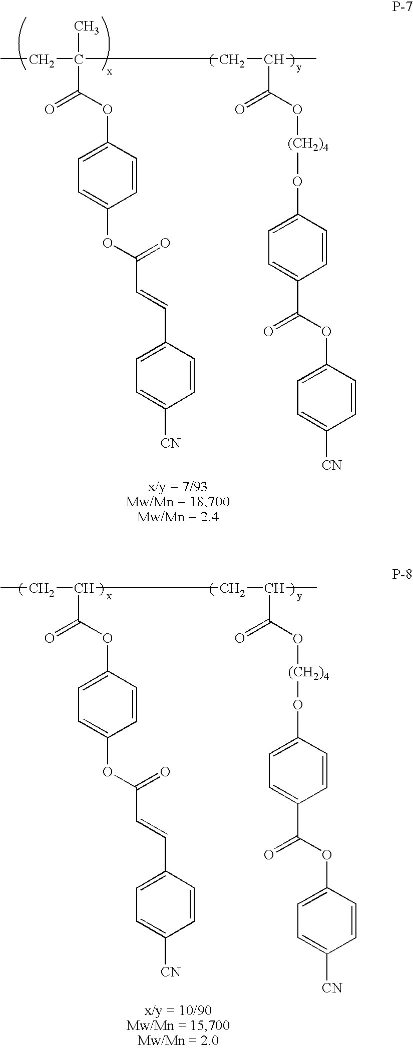

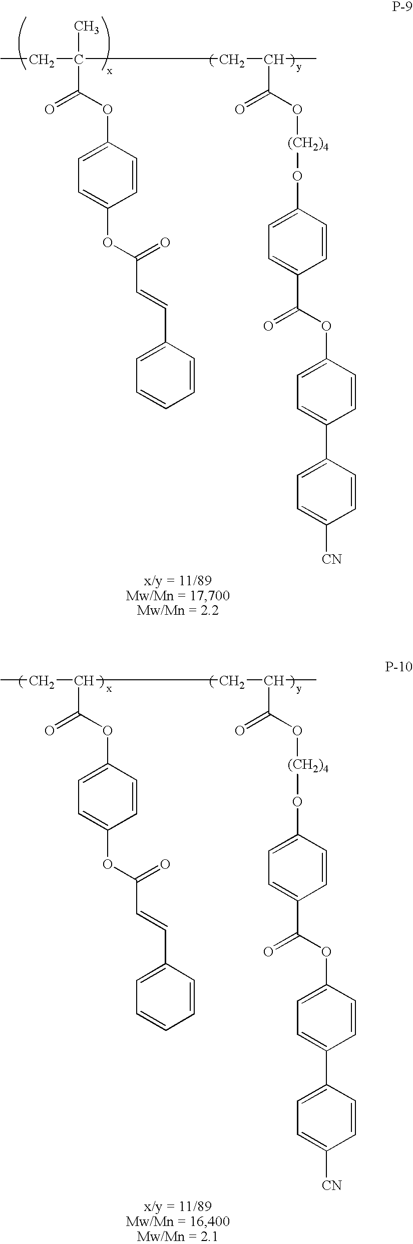

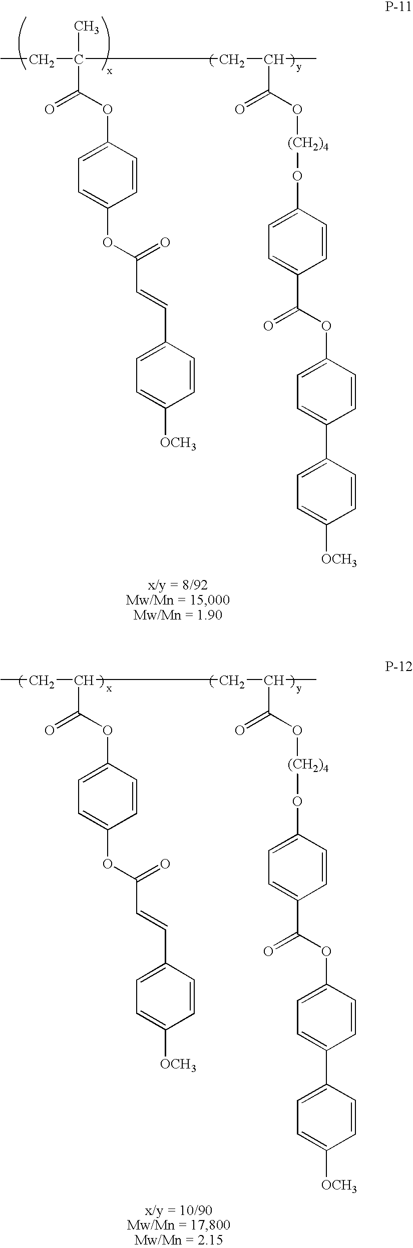

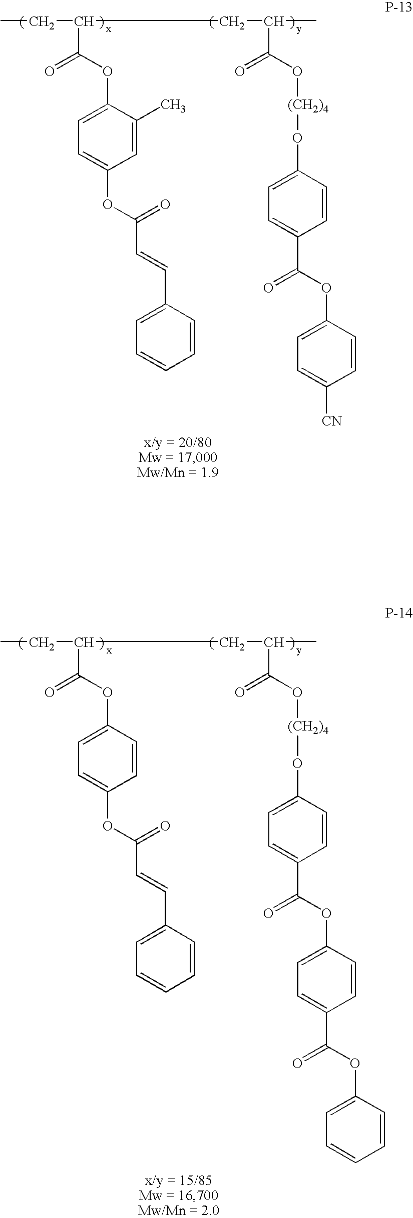

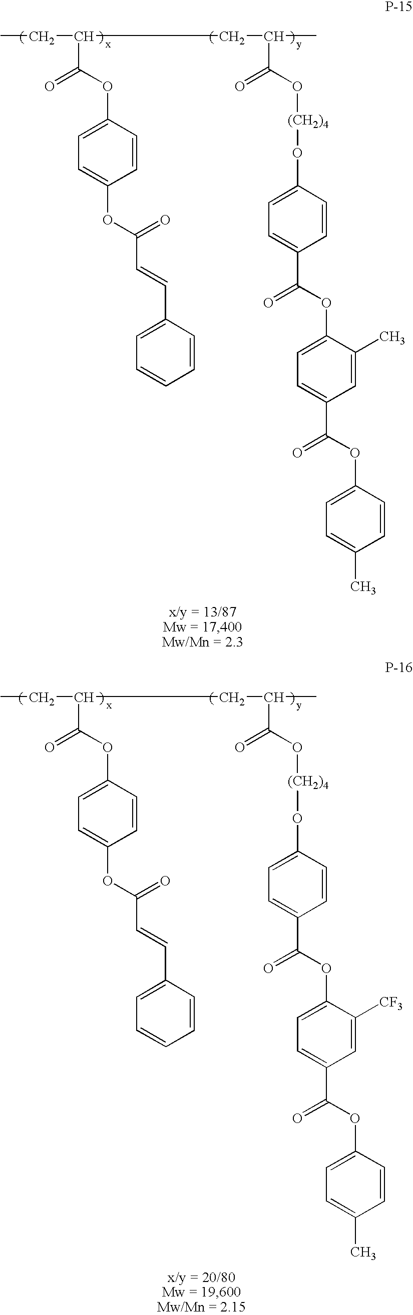

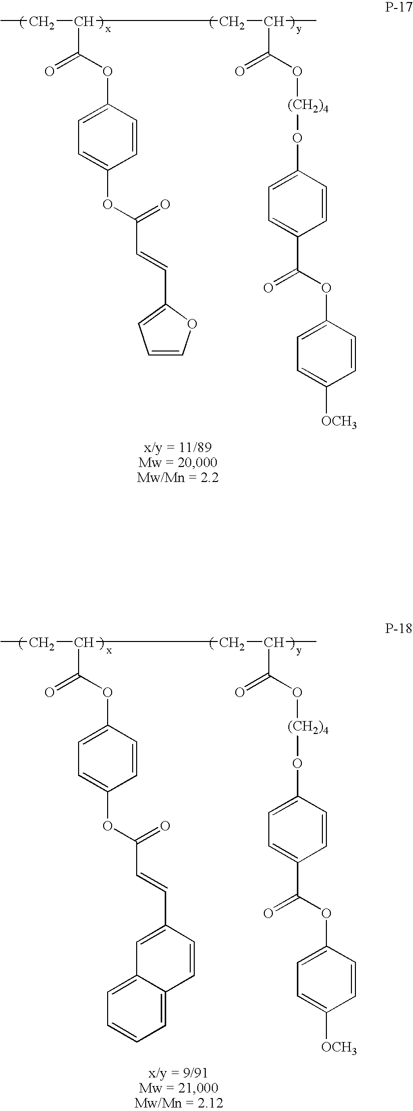

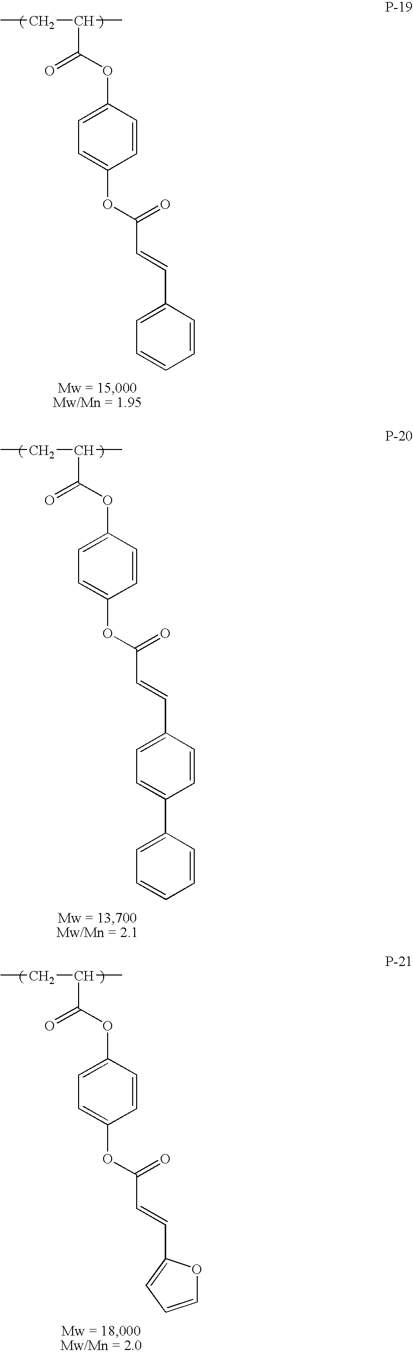

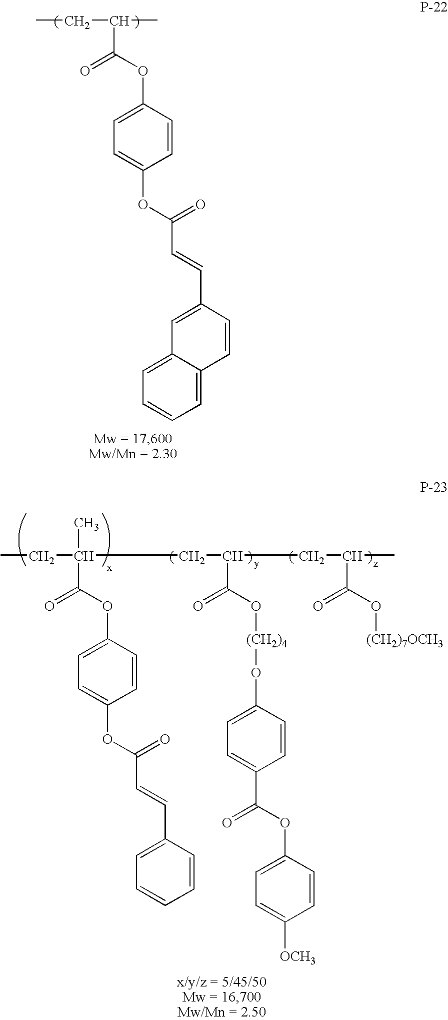

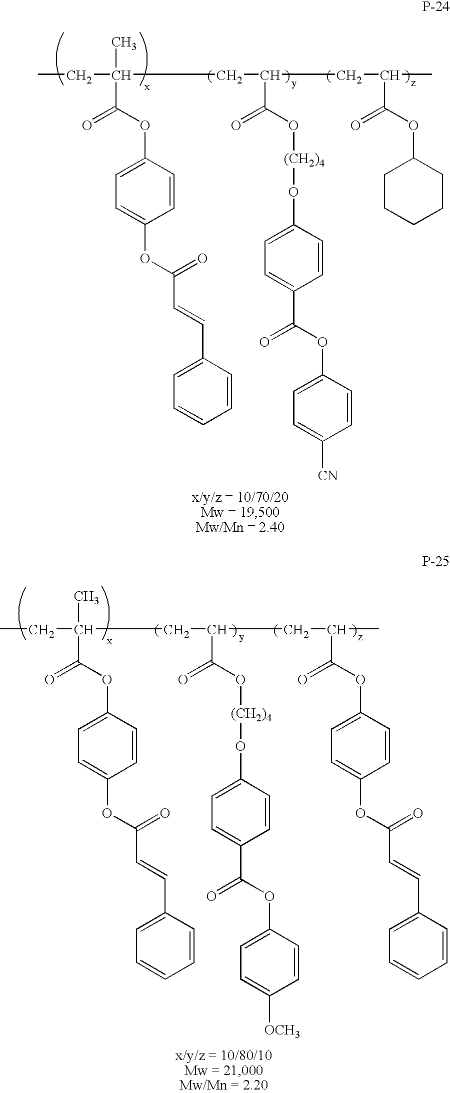

The optically anisotropic film of the invention is formed from the foregoing high molecular compound-containing composition of the invention. In addition to such a composition, various additives can be used jointly, if desired. For example, by using jointly a plasticizer, a surfactant, and the like, it is possible to enhance uniformity of a coating film, strength of a film, orientation properties of the photoreactive group-containing high molecular compound, and the like. A total amount of these additives is preferably not more than 30% by weight, and more preferably not more than 10% by weight relative to the photoreactive group-containing high molecular compound.

Examples of the plasticizer include plasticizers which have hitherto been known. Specific examples thereof include phthalic esters such as dimethyl phthalate, diethyl phthalate, dibutyl phthalate, diisobutyl phthalate, dioctyl phthalate, octylcapryl phthalate, dicyclohexyl phthalate, ditridecyl phthalate, butylbenzyl phthalate, diisodecyl phthalate, and diallyl phthalate; glycol esters such as dimethyl glycol phthalate, ethylphthalylethyl glycolate, methylphthalylethyl glycolate, butylphthalylbutyl glycolate, and triethylene glycol dicaprylate ester; phosphoric esters such as tricresyl phosphate and triphenyl phosphate; aliphatic dibasic acid esters such as diisobutyl adipate, dioctyl adipate, dimethyl sebacate, dibutyl sebacate, dioctyl azelate, and dibutyl maleate; triethyl citrate; glycerin triacetyl ester; and butyl laurate.

Examples of the surfactant include anionic, cationic, nonionic and ampholytic surfactants which have hitherto been known. Fluorine based compounds are especially preferable. Examples thereof include perfluoroalkylamine oxides, perfluoroalkyl ethylene oxide adducts, polymers containing a perfluoroalkyl group and a hydrophilic group, and polymers containing a perfluoroalkyl group, a hydrophilic group and a lipophilic group.

The optically compensatory film of the invention is characterized in that the foregoing optically anisotropic film of the invention is disposed on a substrate. Here, the substrate which is used for the optically compensatory film of the invention is described in detail.

The substrate which is used for the optically compensatory film of the invention is preferably a transparent substrate such as glass and transparent polymer films.

It is preferable that the transparent substrate has a light transmittance of 80% or more. Examples of a polymer which constitutes the polymer film include cellulose esters (for example, cellulose acetate, cellulose propionate, and cellulose butyrate), polycycloolefins (for example, norbornene based polymers), poly(meth)acrylic esters (for example, polymethyl methacrylate), polycarbonates, polyesters, and polysulfones.

As the polymer, cellulose based or cycloolefin based polymers are preferable, and cellulose esters and polycycloolefins are especially preferable. As the cellulose ester, lower fatty acid esters of cellulose are more preferable. The lower fatty acid as referred to herein means a fatty acid having not more than 6 carbon atoms. In particular, cellulose acylates having from 2 to 4 carbon atoms are preferable, and cellulose acetate is more preferable. A mixed fatty acid ester such as cellulose acetate propionate and cellulose acetate butyrate may also be used.

A viscosity-average degree of polymerization (DP) of the cellulose acetate is preferably 250 or more, and more preferably 290 or more. It is preferable that the cellulose acetate has a narrow molecular weight distribution of Mw/Mn (wherein Mw represents a weight-average molecular weight; and Mn represents a number-average molecular weight) by gel permeation chromatography. Specifically, a value of Mw/Mn is preferably from 1.0 to 1.7, and more preferably from 1.0 to 1.65.

As the cellulose acetate, it is preferred to use cellulose acetate having an acetylation degree of from 55.0 to 62.5%; and it is more preferred to use cellulose acetate having a degree of acetylation of from 57.0 to 62.0%.

The acetylation degree as referred to herein means an amount of bound acetic acid per a cellulose unit weight. The degree of acetylation can be determined by measurement of a degree of acetylation in ASTM D-817-91 (testing method for cellulose acetates and the like) and calculation.

As the polycycloolefin, norbornene based polymers are specifically enumerated, and examples thereof include ARTON (manufactured by JSR Corporation) and ZEONOR (manufactured by Zeon Corporation) both of which are a commercially available high molecular compound.

The substrate may have desired optical anisotropy, if desired. A hygroscopic expansion coefficient of the substrate is preferably not more than 30×10⁻⁵/% relative humidity (RH), and more preferably not more than 15×10⁻⁵/% RH. By adjusting this hygroscopic expansion coefficient, it is possible to prevent a frame-shaped increase in the transmittance (light leakage due to a strain) while keeping an optically compensatory function of the optically compensatory film.

The hygroscopic expansion coefficient exhibits an amount of change in the length of a sample when the relative humidity is changed at a fixed temperature. A measurement method of the hygroscopic expansion coefficient is hereunder described.

A specimen having a width of 5 mm and a length of 20 mm is cut out from a prepared polymer film and hung under an atmosphere at 25° C. and 20% RH (R⁰) while fixing one end thereof. After hanging a weight of 0.5 g on the other end, the sample is allowed to stand for 10 minutes and then measured for a length (L⁰).

Next, the humidity is changed to 80% RH (R¹) while keeping the temperature at 25° C. and measured for a length (L¹). The hygroscopic expansion coefficient is calculated according to the following expression. In the measurement, ten samples are used with respect to the same specimen, and an average value of the samples is employed.

Hygroscopic expansion coefficient (/% RH)={(L¹−L⁰)/L⁰}/(R¹−R⁰)

In the polymer film, necessary additives can be added depending upon various purposes. Examples of the additive include an ultraviolet ray preventing agent, a release agent, an antistatic agent, a degradation preventing agent (for example, an antioxidant, a peroxide decomposing agent, a radical inhibitor, a metal inactivating agent, an acid trapping agent, and an amine), and an infra-red ray absorbent. With respect to details thereof, materials described in detail in Kokai Giho of the Japan Institute of Invention and Innovation (Kogi No. 2001-1745, pages 16 to 22, published on Mar. 15, 2001 by the Japan Institute of Invention and Innovation) are preferably used. With respect to an amount of such an additive to be used, the addition amount of each material is not particularly limited so far as the function is exhibited, and it is preferable that the additive is properly used within the range of from 0.001 to 25% by weight in the whole composition of the polymer film. A thickness of the substrate of the invention is preferably from 15 to 120 μm, and more preferably from 30 to 80 μm.

The substrate (polymer film) may be subjected to surface treatment, if desired. Examples of the surface treatment include corona discharge treatment, glow discharge treatment, flame treatment, acid treatment, alkaline treatment, and ultraviolet ray irradiation treatment. These treatments are described in detail in Kokai Giho of the Japan Institute of Invention and Innovation (Kogi No. 2001-1745, pages 30 to 32, published on Mar. 15, 2001 by the Japan Institute of Invention and Innovation).

The optically compensatory film of the invention is configured of the optically anisotropic film layer of the invention and the foregoing substrate. If desired, an orientation layer, a release layer, an adhesive layer, another optically anisotropic layer, and the like may be provided between the subject optically anisotropic layer and the subject substrate.

For the orientation layer, for example, well-known orientation films which are in general used in the field of liquid crystal can be employed. As specific examples of the orientation film, orientation films made of a polyimide, polyvinyl alcohol, or the like and having been subjected to rubbing orientation treatment and orientation films made of an azobenzene derivative, a cinnamic acid derivative, a coumarin derivative, or the like and having been subjected to light orientation treatment can be employed.

For the release layer, well-known release agents and the like can be used. Examples of the release agent include silicon based, phosphoric ester based and fluorine based release agents.

For the adhesive layer, well-known adhesives and the like can be used. Specific examples thereof include acrylic, silicon based and vinyl ether based adhesives. As the adhesive, an optically transparent adhesive is preferable.

For another optically anisotropic layer, for example, optically anisotropic layers constituted of an oriented rod-shaped or disc-shaped liquid crystal compound polymer can be enumerated.

### Optically Anisotropic Film and Method for Producing an Optically Compensatory Film:

The method for producing an optically compensatory film of the invention is a method of coating a composition containing the polymerizable monomer represented by the foregoing Formula (I) on a drum, a band, a substrate or the like to form a high molecular compound layer which makes an optically anisotropic layer and irradiating the high molecular compound layer with light to change an optical characteristic, thereby obtaining a desired optical characteristic. That is, the invention is a method for producing an optically compensatory film comprising of (a) coating a composition containing the foregoing high molecular compound of the invention on the foregoing substrate; and (b) irradiating the substrate with polarized light from a single direction. In the (b), it is also preferable that the polarized light is irradiated on the substrate from an oblique direction. Here, as the polarized light which is used for the irradiation with polarized light, p-polarized light or s-polarized light can be suitably used. Furthermore, it is preferable that the (b) is carried out at a temperature of not higher than a glass transition temperature of the foregoing high molecular compound. Moreover, it is preferable that the production method further includes a step of (c) thermally treating the foregoing substrate and/or the foregoing composition after the (b). According to this method, it is possible to obtain an optically anisotropic film and an optically compensatory film each exhibiting (1) an optical characteristic represented by a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of nx and nz are substantially equal to each other and ny is smaller than nx, (2) an optical characteristic represented by a refractive index ellipsoid obtained by, when a refractive index ellipsoid in which values of nx and nz are substantially equal to each other and ny is smaller than nx is supposed, rotating the subject refractive index ellipsoid around the X-axis as a rotation axis by an arbitrary rotation angle θ, (3) an optical characteristic represented by a refractive index ellipsoid having the relationship of nx>nz>ny, namely an optical characteristic of 0<(nx−nz)/(nx−ny)<1, or (4) an optical characteristic represented by a refractive index ellipsoid obtained by, when a refractive index ellipsoid having the relationship of nx>nz>ny is supposed, rotating the subject refractive index ellipsoid around the foregoing X-axis as a rotation axis by an arbitrary angle θ. By releasing the obtained optically anisotropic film from the drum, the band, the substrate or the like, it can be utilized as a single film. Also, by sticking the released optically anisotropic film with other substrate, it may be utilized as an optically compensatory film; and by transferring the optically anisotropic film from a drum, a band, a substrate or the like into another substrate, it may be utilized as an optically compensatory film. Furthermore, the optically anisotropic film can be utilized as an optically compensatory film as it is without being released from the substrate. The method for producing an optically compensatory film of the invention is hereunder described in detail.

In the (a), a composition (coating liquid) containing the high molecular compound of the invention and other arbitrary components is first coated on a drum, a band, a substrate or the like. As a solvent which is used for preparing the coating liquid, an organic solvent is preferably used. Examples of the organic solvent include amides (for example, N,N-dimethylformamide), sulfoxides (for example, dimethyl sulfoxide), heterocyclic compounds (for example, pyridine), hydrocarbons (for example, benzene and hexane), alkyl halides (for example, chloroform, dichloromethane, and tetrachloroethane), esters (for example, methyl acetate and butyl acetate), ketones (for example, acetone and methyl ethyl ketone), and ethers (for example, tetrahydrofuran and 1,2-dimethoxyethane). Of these, alkyl halides and ketones are preferable. A combination of two or more kinds of organic solvents may also be used.

With respect to the amount of addition of the solvent, its optimal amount is determined within the range where coating properties are not impaired, and it is preferably in the range of from 1 to 75%, and more preferably in the range of from 5 to 50% in terms of the solids content in the coating solvent. Coating of the coating liquid can be carried out by a known method (for example, a spin coating method, a roll coating method, a wire bar coating method, an extrusion coating method, a direct gravure coating method, a reverse gravure coating method, and a die coating method).

Subsequently, the coated material is dried. A known drying method can be employed for drying. Specific examples of the drying method include room temperature drying, heat drying, blast drying, and vacuum drying. These drying methods may be combined with each other.

For the thickness of the optically anisotropic layer, an optimal thickness is applied depending upon the object for use, the form and the optical characteristic. When the optically anisotropic film is used as a single body, its thickness is preferably from 0.1 to 200 μm, and more preferably from 10 to 150 μm. When the optically anisotropic film is used together with the substrate as an optically compensatory film, its thickness is preferably from 0.1 to 200 μm, more preferably from 0.1 to 20 μm, and further preferably from 0.5 to 10 μm.

It is possible to impart an optical characteristic due to the irradiation with light in the (b). The optically anisotropic film and the optically compensatory film of the invention can be obtained through irradiation with polarized light from a single direction from an upper part or a lower part of the substrate.

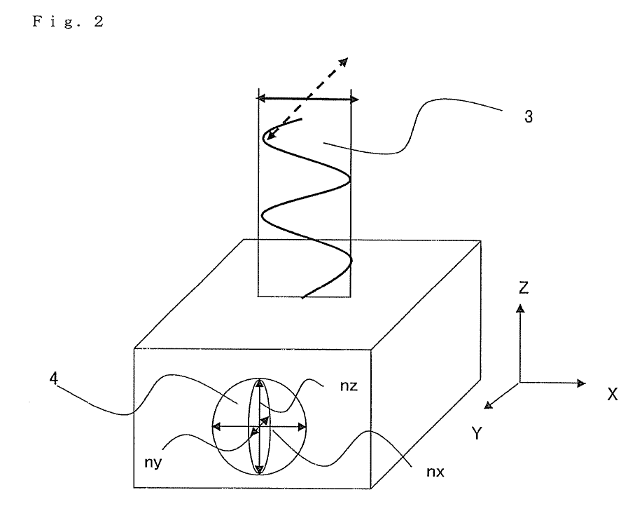

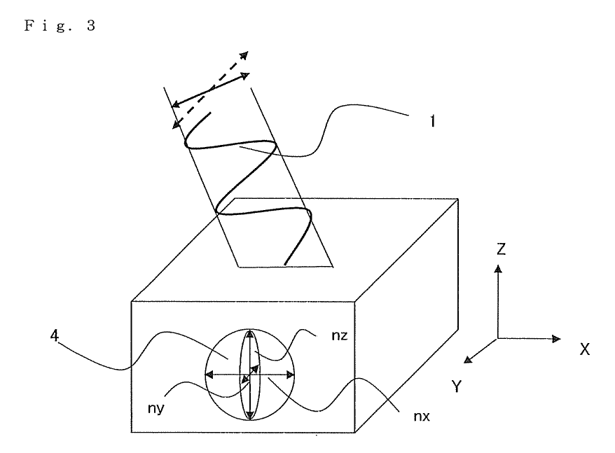

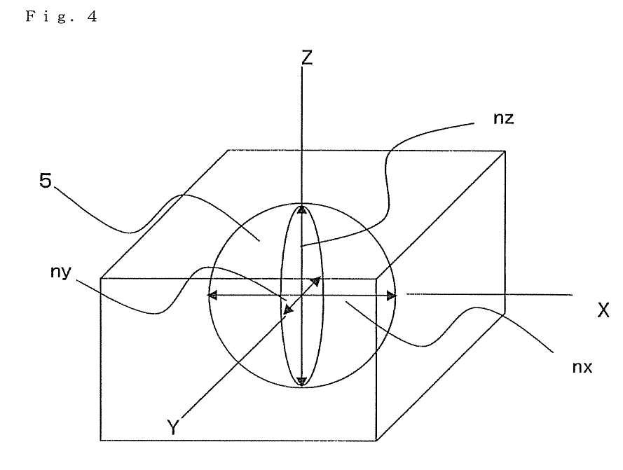

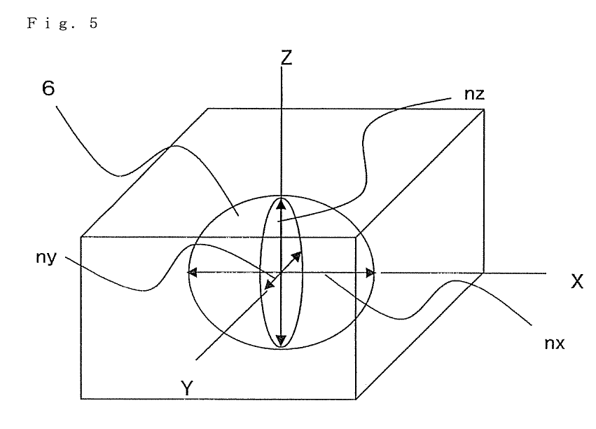

For example, it is possible to obtain an embodiment of the optically anisotropic film and the optically compensatory film of the invention having an optical characteristic represented by a refractive index ellipsoid in which when principal refractive indices in respective axis directions of X, Y and Z against a coordinate system consisting of the X axis and the Y axis in parallel to a film plane and the Z axis in a normal direction of the film plane are defined as nx, ny and nz, respectively, values of nx and nz are substantially equal to each other and ny is smaller than nx (FIG. 4) or an optical characteristic represented by a refractive index ellipsoid having the relationship of nx>nz>ny (FIG. 5) by irradiating the high molecular compound layer composed of a high molecular compound containing a repeating unit derived from a monomer represented by the foregoing Formula (I) with polarized light from a vertical direction to the substrate (FIG. 2), or by irradiating it with p-polarized light from an oblique direction to the substrate (FIG. 3).

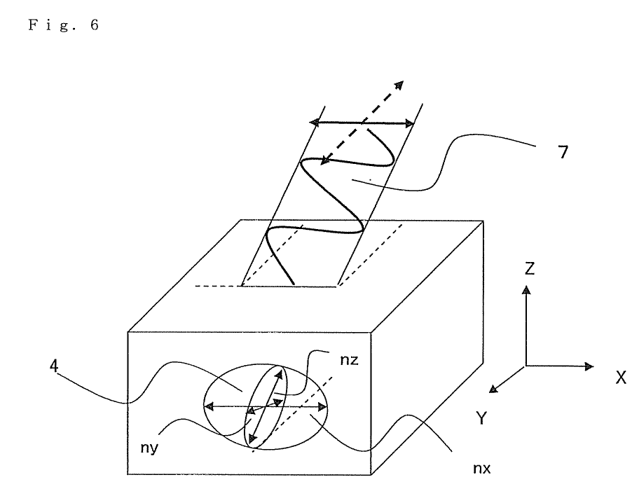

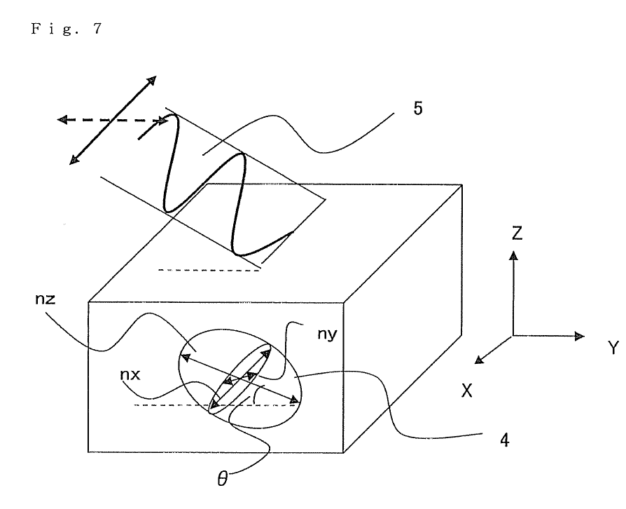

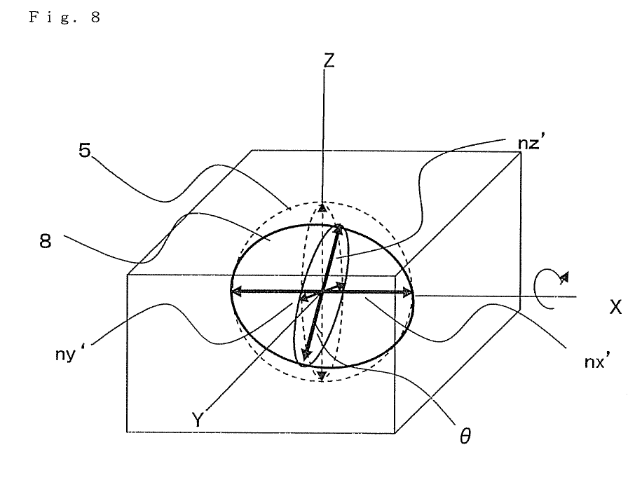

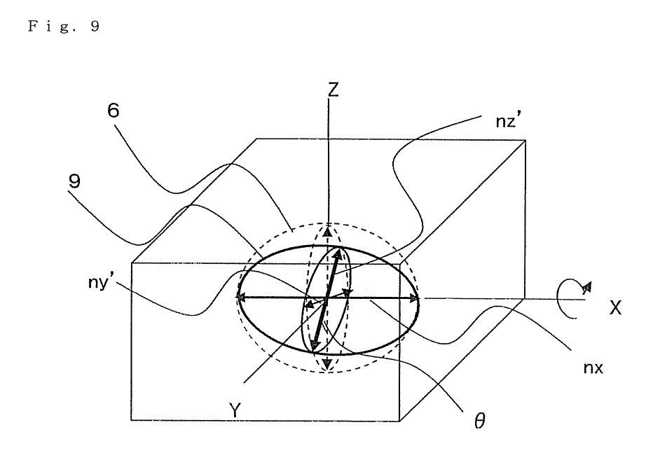

For example, it is possible to obtain another embodiment of the optically anisotropic film and the optically compensatory film of the invention having an optical characteristic represented by a refractive index ellipsoid obtained by, when in the case where principal refractive indices in respective axis directions of X, Y and Z against a coordinate system consisting of the X axis and the Y axis in parallel to a film plane and the Z axis in a normal direction of the film plane are defined as nx, ny and nz, respectively, a refractive index ellipsoid in which values of nx and nz are substantially equal to each other and ny is smaller than nx is supposed, rotating the refractive index ellipsoid around the X-axis as a rotation axis by an arbitrary rotation angle θ (FIG. 8) or an optical characteristic represented by a refractive index ellipsoid obtained by, when a refractive index ellipsoid having the relationship of nx>nz>ny is supposed, rotating the refractive index ellipsoid around the X-axis as a rotation axis by an arbitrary rotation angle θ (FIG. 9) by irradiating the high molecular compound layer composed of a high molecular compound containing a repeating unit derived from a monomer represented by the foregoing Formula (I) with s-polarized light from an oblique direction to the substrate (FIGS. 6 and 7).

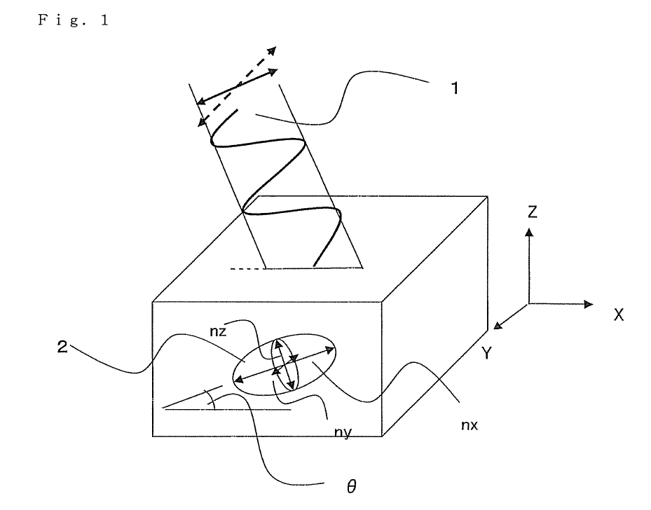

In FIGS. 1 to 9, 1 represents p-polarized light; 2 a refractive index ellipsoid; 3 linear polarized light; 4 a refractive index ellipsoid having the relationship of nx=nz and nx>ny, or nx>nz>ny; 5 a refractive index ellipsoid having the relationship of nx=nz and nx>ny; 6 a refractive index ellipsoid having the relationship of nx>nz>ny; 7 s-polarized light; 8 a refractive index ellipsoid obtained by rotating a refractive index ellipsoid having the relationship of nx=nz and nx>ny around the X-axis as a rotation axis by an arbitrary rotation angle θ; and 9 a refractive index ellipsoid obtained by rotating a refractive index ellipsoid having the relationship of nx>nz>ny around the X-axis as a rotation axis by an arbitrary rotation angle θ, respectively.

Examples of the light to be irradiated include X-rays, electron beams, ultraviolet rays, visible light, and infrared rays (heat rays), with ultraviolet rays being preferable. A wavelength of the ultraviolet rays is preferably not longer than 400 nm, and a peak wavelength thereof is more preferably from 250 nm to 360 nm. As a light source, a low-pressure mercury lamp, a high-pressure discharge lamp, and a short-arc discharge lamp are preferably used.

As a measure for obtaining polarized light, a method of using a polarizing plate (for example, an iodine polarizing plate, a dichroic dye polarizing plate, and a wire grid polarizing plate), a method of using a prism based device (for example, a Glan-Thomson prism) or a reflection type polarizer utilizing a Brewster's angle, or a method of using light outputted from a laser light source with polarized light can be employed. Only light having a necessary wavelength may be selectively irradiated by using a filter, a wavelength modulation device, or the like.

It is possible to adjust the resulting optical characteristic depending upon irradiation intensity, irradiation time and irradiation temperature of the irradiation with light and drying condition of the high molecular compound layer at the irradiation. Accordingly, with respect to the irradiation intensity, the irradiation time, the irradiation temperature and the drying condition of the high molecular compound at the irradiation, an optimal condition is employed depending upon the desired optical characteristic and the high molecular compound to be used.

The irradiation intensity and irradiation time of the irradiation with light can be adjusted such that they are optimal in conformity with the desired optical characteristic depending upon the high molecular compound to be used. An irradiation dose is preferably from 10 mJ/cm² to 30,000 mJ/cm², and more preferably from 20 mJ/cm² to 6,000 mJ/cm².

The temperature at the irradiation with light can be adjusted such that it is optimal in conformity with the desired optical characteristic depending upon the high molecular compound to be used and is preferably not higher than Tg of the high molecular compound to be used. In particular, for the purpose of obtaining an optical characteristic represented by a refractive index ellipsoid in which when principal refractive indices in respective axis directions of X, Y and Z are defined as nx, ny and nz, respectively, values of nx and nz are substantially equal to each other and ny is smaller than nx; and an optical characteristic represented by a refractive index ellipsoid obtained by, when a refractive index ellipsoid in which values of nx and nz are substantially equal to each other and ny is smaller than nx is supposed, rotating the refractive index ellipsoid around the X-axis as a rotation axis by an arbitrary rotation angle θ, the temperature at the irradiation with light is preferably not higher than Tg of the high molecular compound to be used, and preferably not higher than room temperature.

By thermally treating the thus-obtained optically anisotropic film, it is possible to adjust the optical characteristic. The heating temperature and heating time can be adjusted such that they are optimal in conformity with the desired optical characteristic depending upon the high molecular compound to be used. By applying heating, it is possible to increase a value of (nx−nz)/(nx−ny).

By releasing the thus-obtained optically anisotropic film from the drum, the band, the substrate or the like, it is possible to obtain the optically anisotropic film of the invention. Also, by sticking the released optically anisotropic film with other substrate or transferring it from a drum, a band, a substrate or the like into another substrate, it is possible to obtain the optically compensatory film. Furthermore, the optically anisotropic film can be utilized as an optically compensatory film as it is without being released from the substrate.

### <Application of Optically Compensatory Film>

For example, the optically compensatory film of the invention can be provided for an application to a polarizing plate (elliptical polarizing plate) through combination with a polarizing film. The optically compensatory film of the invention contributes to enlargement of a viewing angle by combining with a polarizing film and applying the combination to a transmission type liquid crystal display device. The polarizing plate of the invention is a polarizing plate comprising a polarizer interposed between two protective films, wherein at least one of the two protective films is the foregoing optically anisotropic film or optically compensatory film of the invention. The liquid crystal display device of the invention comprises the foregoing optically anisotropic film or optically compensatory film of the invention. An elliptical polarizing plate and a liquid crystal display device each utilizing the optically compensatory film of the invention are hereunder described in detail.

### [Elliptical Polarizing Plate]

The foregoing elliptical polarizing plate can be prepared by stacking the optically compensatory film of the invention and a polarizing film. By utilizing the optically compensatory film of the invention, it is possible to provide an elliptical polarizing plate capable of enlarging a viewing angle of a liquid crystal display device. Examples of the foregoing polarizing film include an iodine based polarizing film, a dye based polarizing film using a dichroic dye, and a polyene based polarizing film. The iodine based polarizing film and the dye based polarizing film can be in general manufactured by using a polyvinyl alcohol based film. An axis of polarization of the polarizing film is corresponding to a vertical direction to a stretch direction of the film.

The foregoing polarizing film is stacked on a side of the optically anisotropic layer of the foregoing optically compensatory film. It is preferable that a protective film is formed on a surface on an opposite side to the side on which the optically compensatory film of the polarizing film is stacked. The protective film is preferably a protective film (transparent protective film) having a light transmittance of 80% or more. As the transparent protective film, a cellulose ester film is preferably used, and a triacetyl cellulose film is more preferably used. It is preferable that the cellulose ester film is formed by a solvent casting method. A thickness of the protective film is preferably from 20 to 500 μm, and more preferably from 50 to 200 μm.

### [Liquid Crystal Display Device]

By utilizing the optically compensatory film of the invention, it is possible to provide a liquid crystal display device with an enlarged viewing angle. Also, it is possible to provide a liquid crystal display device capable of displaying a high-quality image which is free from display unevenness. As an optically compensatory film for liquid crystal cell of a Twisted Nematic (TN) mode, the optically compensatory film of the invention can be utilized according to descriptions of, for example, JP-A-6214116, U.S. Pat. Nos. 5,583,679 and 5,646,703, and German Patent No. 3911620A1. As an optically compensatory film for liquid crystal cell of an In-plane Switching (IPS) mode or a Ferroelectric Liquid Crystal (FLC) mode, the optically compensatory film of the invention can be utilized according to a description of JP-A-10-54982. As an optically compensatory film for liquid crystal cell of an Optically Compensatory Bend (OCB) mode or a Hybrid Aligned Nematic (HAN) mode, the optically compensatory film of the invention can be utilized according to descriptions of U.S. Pat. No. 5,805,253, WO 96/37804, and the like. As an optically compensatory film for liquid crystal cell of a Super Twisted Nematic (STN) mode, the optically compensatory film of the invention can be utilized according to a description of JP-A-9-26572. As an optically compensatory film for liquid crystal cell of a Vertically Aligned (VA) mode, the optically compensatory film of the invention can be utilized according to a description of Japanese Patent No. 2866372.

### EXAMPLES

The invention is more specifically described below with reference to the Examples. Materials, amounts of use, proportions, contents of treatment, treatment procedures, and the like as described in the following Examples can be properly changed so far as the gist of the invention is not deviated. Accordingly, it should be construed that the scope of the invention is not limited to these specific examples.

### Example 1

### Synthesis of Polymerizable Monomer (A-14: 4-methacryloyloxyphenylester cinnamate)

1. Synthesis of 4-hydroxyphenylestel methacrylate:

134 g of hydroquinone and 54 mL of triethylamine were added to 1.2 L of tetrahydrofuran (THF) and stirred at 0° C. for one hour; 31.4 g of methacrylic acid chloride was gradually added dropwise to this mixture; and stirring was further continued at 0° C. for 3 hours. 1 L of ethyl acetate and 1 L of pure water were added to the resulting solution, and the mixture was stirred. The ethyl acetate layer was subjected to liquid separation, washed with a saturated salt aqueous solution and then dried over anhydrous magnesium sulfate. The magnesium sulfate was filtered off, and the solution was concentrated and purified by means of silica chromatography, thereby obtaining 32 g of 4-hydroxyphenylester methacrylate.

¹H-NMR (CDCl₃, δ ppm): 6.95 (2H, d), 6.75 (2H, d), 6.3 (1H, s), 5.75 (1H, s), 5.2 (1H, s), 2.05 (3H, s)

2. Synthesis of 4-methacryloyloxyphenylester cinnamate:

14 g of the 4-hydroxyphenylester methacrylate as synthesized above and 22 mL of triethylamine were added to 200 mL of THF and stirred at 0° C. for one hour. Next, 14.5 g of cinnamic acid chloride was dissolved in 50 mL of THF and added dropwise. Stirring was further continued at 0° C. for 3 hours; 0.5 L of ethyl acetate and 0.5 L of pure water were added to the resulting solution; and the mixture was stirred. The ethyl acetate layer was subjected to liquid separation, washed with a saturated salt aqueous solution and then dried over anhydrous magnesium sulfate. The magnesium sulfate was filtered off; and the solution was concentrated, separated by means of silica chromatography and then recrystallized from hexane/ethyl acetate, thereby obtaining 11 g of 4-methacryloyloxyphenylester cinnamate.

¹H-NMR (CDCl₃, δ ppm): 7.9 (2H, d), 7.6 (2H, dd), 7.5 (3H, m), 7.2 (4H, m), 6.6 (1H, d), 6.35 (1H, s), 5.75 (1H, s), 2.05 (3H, s)

### Example 2

### Synthesis of Polymerizable Monomer (4-methacryloyloxyphenylester 4-phenylcinnamate)

14 g of the 4-hydroxyphenylester methacrylate as synthesized in the foregoing Example 1 and 22 mL of triethylamine were added to 200 mL of THF and stirred at 0° C. for one hour. Next, 21 g of 4-phenylcinnamic acid chloride was dissolved in 50 mL of THF and added dropwise. Stirring was further continued at 0° C. for 3 hours; 0.5 L of ethyl acetate and 0.5 L of pure water were added to the resulting solution; and the mixture was stirred. The ethyl acetate layer was subjected to liquid separation, washed with a saturated salt aqueous solution and then dried over anhydrous magnesium sulfate. The magnesium sulfate was filtered off; and the solution was concentrated, separated by means of silica chromatography and then recrystallized from ethyl acetate, thereby obtaining 16 g of 4-methacryloyloxyphenyl 4-cinnamate phenyl ester.

¹H-NMR (CDCl₃, δ ppm): 7.9 (2H, d), 7.65 (6H, m), 7.5 (2H, m), 7.4 (2H, d), 7.2 (4H, m), 6.65 (1H, d), 6.35 (1H, s), 5.75 (1H, s), 2.05 (3H, s)

### Example 3

### Synthesis of Polymerizable Monomer (A-1: 4-acryloyloxyphenylester cinnamate)

1. Synthesis of 4-hydroxyphenylester acrylate:

134 g of hydroquinone and 54 mL of triethylamine were added to 1.2 L of THF and stirred at 0° C. for one hour; 27.2 g of acrylic acid chloride was gradually added dropwise to this mixture; and stirring was further continued at 0° C. for 3 hours. 1 L of ethyl acetate and 1 L of pure water were added to the resulting solution, and the mixture was stirred. The ethyl acetate layer was subjected to liquid separation, washed with a saturated salt aqueous solution and then dried over anhydrous magnesium sulfate. The magnesium sulfate was filtered off, and the solution was concentrated and purified by means of silica chromatography, thereby obtaining 26 g of 4-hydroxyphenylester acrylate.

¹H-NMR (CDCl₃, δ ppm): 7.0 (2H, d), 6.8 (2H, d), 6.6 (1H, dd), 6.35 (1H, dd), 6.0 (1H, d), 5.2 (1H, s)

2. Synthesis of 4-acryloyloxyphenylester cinnamate:

13 g of the 4-hydroxyphenylester acrylate as synthesized above and 22 mL of triethylamine were added to 200 mL of THF and stirred at 0° C. for one hour. Next, 14.5 g of cinnamic acid chloride was dissolved in 50 mL of THF and added dropwise. Stirring was further continued at 0° C. for 3 hours; 0.5 L of ethyl acetate and 0.5 L of pure water were added to the resulting solution; and the mixture was stirred. The ethyl acetate layer was subjected to liquid separation, washed with a saturated salt aqueous solution and then dried over anhydrous magnesium sulfate. The magnesium sulfate was filtered off; and the solution was concentrated, separated by means of silica chromatography and then recrystallized from hexane/ethyl acetate, thereby obtaining 13 g of 4-acryloyloxyphenyl cinnamate. The analytical results are shown below.

¹H-NMR (CDCl₃, δ ppm): 7.9 (1H, d), 7.6 (2H, dd), 7.4 (3H, m), 7.2 (4H, m), 6.6 (2H, m), 6.35 (1H, dd), 6.0 (1H, d)

### Synthesis Example 1

A compound P-1 of the invention was synthesized by radical polymerization of 9 parts (molar ratio) of the photoreactive group-containing polymerizable monomer (A-14) and 1 part (molar ratio) of a monomer (M-11) under nitrogen at 70° C. for 10 hours using 2,2-azobisisobutyronitrile (2% by mole) as an initiator and anhydrous N,N-dimethylacetylamide as a solvent, followed by purification by means of reprecipitation from methanol.

P-1 had a weight-average molecular weight as measured by GPC of 18,000, Tg as measured by DSC of 38.4° C. and an x/y molar ratio as measured by H-NMR of 5/95.

### Synthesis Example 2

A high molecular compound P-2 was synthesized in the same manner as in the foregoing Synthesis Example 1 except for using 1 part (molar ratio) of A-1 and 9 parts (molar ratio) of M-11. x=11, y=89, Mw=16,800, Tg=39° C.

### Synthesis Example 3

Compounds P-3 to P-25 were synthesized in the similar manner as in the foregoing Synthesis Example 1.

### Example 4

A coating liquid prepared by dissolving 3 g of the foregoing illustrative compound P-1 (x=5, y=95, Mw=18,000, Tg=38.4° C.) as the high molecular compound of the invention in 10 g of tetrahydrofuran was coated on a glass substrate having a thickness of 1.1 mm as a substrate by a spin coating method. After drying at room temperature for 120 seconds, light outputted from ultraviolet rays which were outputted from an ultraviolet ray irradiator (EXECURE 3000, manufactured by Hoya Candeo Optronics Corporation) was converted into linear polarized light via a polarizing plate, which was then irradiated at an intensity of 100 mW (365 nm) for 300 seconds from a vertical direction to the substrate, thereby preparing an optically compensatory film having an optically anisotropic film on the glass substrate. At that time, the optically anisotropic layer had a thickness of 2.0 μm.

### Examples 5 to 7

Optically compensatory films were prepared in the same manner as in Example 4, except that in the preparation of a high molecular compound composition coating liquid, the photoreactive group-containing high molecular compound P-1 was changed to P-2 (x=11, y=89, Mw=6,800, Tg=39° C.), P-3 (x=7, y=93, Mw=19,400, Tg=40° C.) and P-5 (x=5, y=95, Mw=16,700, Tg=40° C.), respectively. At that time, the optically anisotropic layers had a thickness of 3.1 μm, 3.3 μm and 2.0 μm, respectively.

### Example 8

A coating liquid prepared by dissolving 3 g of the foregoing illustrative compound P-2 (x=11, y=89, Mw=6,800, Tg=39° C.) as the photoreactive group-containing high molecular compound in 10 g of tetrahydrofuran was coated on a glass substrate having a thickness of 1.1 mm as a substrate by a spin coating method. After drying at room temperature for 120 seconds, light outputted from ultraviolet rays which were outputted from an ultraviolet ray irradiator (EXECURE 3000, manufactured by Hoya Candeo Optronics Corporation) was converted into linear polarized light via a polarizing plate, and p-polarized light was then irradiated at an intensity of 100 mW (365 nm) for 300 seconds from a direction inclined by 45° from the vertical direction to the substrate, thereby preparing an optically compensatory film having an optically anisotropic film on the glass substrate. At that time, the optically anisotropic layer had a thickness of 2.0 μm.

### Referential Example 1

An optically compensatory film was prepared in the same manner as in Example 4, except that in the formation of an optically anisotropic layer, the irradiation with light was not carried out. At that time, the optically anisotropic layer had a thickness of 2.0 μm.

An optical characteristic of each of the optically compensatory films as prepared in Examples 4 to 8 and Referential Example 1 was measured by using KOBRA WR (manufactured by Oji Scientific Instruments), and its principal refractive indices nx, ny and nz were calculated by a software N-Calc for calculation of three-dimensional refractive index (manufactured by Oji Scientific Instruments). The results are shown in Table 1.

TABLE 1

| | Photoreactive | | | | | |
|---|---|---|---|---|---|---|
| | group-containing | | | | | |
| | high molecular | Re | | | | (nx − nz)/ |
| | compound | (nm) | nx | ny | nz | (nx − ny) |
| Example 4 | P-1 | 129.8 | 1.623 | 1.558 | 1.618 | 0.08 |
| Example 5 | P-2 | 53 | 1.610 | 1.583 | 1.607 | 0.11 |
| Example 6 | P-3 | 96.4 | 1.611 | 1.580 | 1.609 | 0.06 |
| Example 7 | P-5 | 143.7 | 1.614 | 1.571 | 1.613 | 0.02 |
| Example 8 | P-2 | 109.9 | 1.620 | 1.565 | 1.615 | 0.09 |
| Referential | P-1 | 0 | 1.601 | 1.600 | 1.599 | — |
| Example 1 | | | | | | |

From the foregoing results, it has become clear that when the photoreactive group-containing high molecular compound of the invention is used, by irradiating the substrate with polarized light from the vertical direction or irradiating the substrate with p-polarized light from a direction at an arbitrary angle from the vertical direction, an optical characteristic represented by a refractive index ellipsoid in which values of nx and nz are substantially equal to each other and ny is smaller than nx is obtained. Furthermore, from the results of Example 4 and Referential Example 1, an optically compensatory film in which an optical characteristic of the optically anisotropic layer is changed upon irradiation with ultraviolet rays, larger retardation can be exhibited and a desired optical characteristic is brought upon irradiation with light could be obtained.

### Example 9

A coating liquid prepared by dissolving 3 g of the foregoing illustrative compound P-2 (x=11, y=89, Mw=6,800, Tg=39° C.) as the high molecular compound of the invention in 10 g of tetrahydrofuran was coated on a glass substrate having a thickness of 1.1 mm as a substrate by a spin coating method. After drying at room temperature for 120 seconds, light outputted from ultraviolet rays which were outputted from an ultraviolet ray irradiator (EXECURE 3000, manufactured by Hoya Candeo Optronics Corporation) was converted into linear polarized light via a polarizing plate, and s-polarized light was then irradiated at an intensity of 100 mW (365 nm) for 300 seconds from a direction inclined by an arbitrary angle from the vertical direction to the substrate, thereby preparing an optically compensatory film having an optically anisotropic film on the glass substrate.

An optical characteristic of the prepared optically compensatory film was measured by using KOBRA WR (manufactured by Oji Scientific Instruments), and on the assumption that a refractive index ellipsoid was disc-shaped, β calculation was carried out by using KOBRA WR, thereby calculating an average inclination of the refractive index ellipsoid in the optically compensatory film. Table 2 shows the relationship between an incident angle of the s-polarized light (an angle at the vertical incidence is defined as 0°) and an average inclination of the obtained refractive index ellipsoid.

From the foregoing results, it has become clear that when the photoreactive group-containing high molecular compound of the invention is used, by irradiating the substrate with s-polarized light from a direction at an arbitrary angle from the vertical direction, an optical characteristic represented by a refractive index ellipsoid obtained by, when in the case where principal refractive indices in respective axis directions of X, Y and Z against a coordinate system consisting of the X axis and the Y axis in parallel to a film plane and the Z axis in a normal direction of the film plane are defined as nx, ny and nz, respectively, a refractive index ellipsoid in which values of nx and nz are substantially equal to each other and ny is smaller than nx is supposed, rotating the refractive index ellipsoid around the X-axis as a rotation axis by an arbitrary rotation angle θ is obtained, and an optically compensatory film having a desired optical characteristic upon irradiation with light could be obtained.

TABLE 2

| | Incident angle of | | | | | | | | |
|---|---|---|---|---|---|---|---|---|---|
| | s-polarized light | | | | | | | | |
| | (• •ᵢₙ) | | | | | | | | |
| | 0° | 10° | 20° | 30° | 40° | 50° | 60° | 70° | 80° |
| Average inclination | 90° | 85° | 80° | 73° | 67° | 62° | 59° | 54° | 52° |
| of refractive index | | | | | | | | | |
| ellipsoid | | | | | | | | | |

### Example 10

The optically compensatory film obtained in Example 9 was thermally treated at 50° C.; a change in an optical characteristic of the optically compensatory film against the heating time was measured by using KOBRA WR (manufactured by Oji Scientific Instruments), and its principal refractive indices nx, ny and nz were calculated by a software N-Calc for calculation of three-dimensional refractive index (manufactured by Oji Scientific Instruments). The results are shown in Table 3. From the foregoing results, it has become clear that a value of (nx−nz)/(nx−ny) can be increased by means of heating, and an optically compensatory film having a desired optical characteristic by heating could be obtained.

TABLE 3

| | Re | | | | (nx − nz)/ |
|---|---|---|---|---|---|
| Heating time | (nm) | nx | ny | nz | (nx − ny) |
| Example 9 | 53 | 1.610 | 1.583 | 1.607 | 0.11 |
| 15 minutes | 74.1 | 1.617 | 1.580 | 1.602 | 0.27 |
| 60 minutes | 77.2 | 1.618 | 1.580 | 1.602 | 0.42 |
| 120 minutes | 79.1 | 1.619 | 1.580 | 1.601 | 0.46 |
| 180 minutes | 83.8 | 1.621 | 1.580 | 1.599 | 0.54 |

### Example 11

A coating liquid prepared by dissolving 3 g of the foregoing illustrative compound P-2 (x=11, y=89, Mw=6,800, Tg=39° C.) as the photoreactive group-containing high molecular compound in 10 g of tetrahydrofuran was coated on a glass substrate having a thickness of 1.1 mm as a substrate by a spin coating method. After drying at room temperature for 120 seconds, light outputted from ultraviolet rays which were outputted from an ultraviolet ray irradiator (EXECURE 3000, manufactured by Hoya Candeo Optronics Corporation) was converted into linear polarized light via a polarizing plate, and s-polarized light was then irradiated at an intensity of 100 mW (365 nm) for 300 seconds from a direction inclined by 45° from the vertical direction to the substrate, thereby preparing an optically compensatory film having an optically anisotropic film on the glass substrate. Next, heat treatment was carried out 50° C. for 10 minutes.

An optical characteristic of the prepared optically compensatory film was measured by KOBRA WR (manufactured by Oji Scientific Instruments), and on the assumption that a refractive index ellipsoid was disc-shaped, β calculation was carried out by using KOBRA WR, thereby calculating an average inclination of the refractive index ellipsoid in the optically compensatory film. As a result, a value of 66° was obtained.

From the foregoing results, it has become clear that when the photoreactive group-containing high molecular compound of the invention is used, by irradiating the substrate with s-polarized light from a direction at an arbitrary angle from the vertical direction, an optical characteristic represented by a refractive index ellipsoid obtained by, when in the case where principal refractive indices in respective axis directions of X, Y and Z against a coordinate system consisting of the X axis and the Y axis in parallel to a film plane and the Z axis in a normal direction of the film plane are defined as nx, ny and nz, respectively, a refractive index ellipsoid having the relationship of nx>nz>ny is supposed, rotating the refractive index ellipsoid around the X-axis as a rotation axis by an arbitrary rotation angle θ is obtained.

### Example 12

A coating liquid prepared by dissolving 1 g of the foregoing illustrative compound P-2 (x=11, y=89, Mw=6,800, Tg=39° C.) as the photoreactive group-containing high molecular compound in 10 g of methyl ethyl ketone was coated on a commercially available cellulose acetate film, FUJITAC TD80UF (manufactured by Fuji Photo Film Co., Ltd., Re=3 nm, Rth=50 nm) as a substrate by a wire bar coating method. After drying at room temperature for 120 seconds, light outputted from ultraviolet rays which were outputted from an ultraviolet ray irradiator (EXECURE 3000, manufactured by Hoya Candeo Optronics Corporation) was converted into linear polarized light via a polarizing plate, and s-polarized light was then irradiated at an intensity of 100 mW (365 nm) for 300 seconds from a vertical direction to the substrate, thereby preparing an optically compensatory film having an optically anisotropic film on the cellulose acetate film.

According to the invention, it has become possible to obtain an optically anisotropic film having an optical characteristic which is in general difficultly prepared. In particular, according to the invention, it is possible to provide a novel optically compensatory film in which by changing an irradiation condition such as irradiation angle and irradiation intensity of light, complicated refractive index anisotropy can be more precisely controlled and which is excellent in an optically compensatory function and when applied to an image display device, contributes to enlargement in the viewing angle.

The present disclosure relates to the subject matter contained in Japanese Patent Application No. 226888/2006 filed on Aug. 23, 2006, which is expressly incorporated herein by reference in their entirety. All the publications referred to in the present specification are also expressly incorporated herein by reference in their entirety.

The foregoing description of preferred embodiments of the invention has been presented for purposes of illustration and description, and is not intended to be exhaustive or to limit the invention to the precise form disclosed. The description was selected to best explain the principles of the invention and their practical application to enable others skilled in the art to best utilize the invention in various embodiments and various modifications as are suited to the particular use contemplated. It is intended that the scope of the invention not be limited by the specification, but be defined claims set forth below.

1. A polymerizable monomer represented by the following Formula (I): 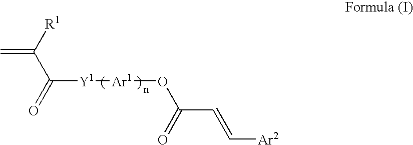
wherein R¹ represents a hydrogen atom or a substituted group; Y¹ represents an oxygen atom or —NR³—, wherein R³ represents a hydrogen atom or an alkyl group; Ar¹ and Ar² each independently represents an aromatic ring having from 1 to 10 carbon atoms, and each of Ar¹ and Ar² may have a substituted group; and n represents an integer of from 1 to 3.

2. The polymerizable monomer according to claim 1, wherein Ar¹ is an aromatic ring represented by the following Formula (II): 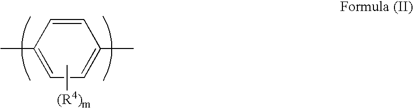
wherein R⁴ represents a substituted group; m represents an integer of from 0 to 4; and when m is an integer of from 2 to 4, plural R⁴s may be the same or different.

3. The polymerizable monomer according to claim 1, wherein Ar² is an optionally substituted benzene ring, furan ring or naphthalene ring.

4. A high molecular compound formed of the polymerizable monomer according to claim 1.

5. The high molecular compound according to claim 4, formed by using further a polymerizable monomer represented by the following Formula (III): 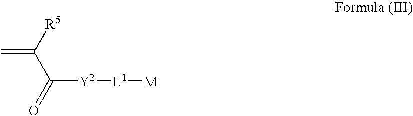
wherein R⁵ represents a hydrogen atom or a substituted group; L¹ represents a single bond or a divalent linking group; M represents a mesogen; and Y² represents —NR⁶—, wherein R⁶ represents an alkyl group or a hydrogen atom, or —O—.

6. An optically anisotropic film formed of a composition containing the high molecular compound according to claim 4.

7. An optically compensatory film comprising a substrate having thereon the optically anisotropic film according to claim 6.

8. The optically compensatory film according to claim 7, wherein the substrate comprises a cellulose-based polymer or a cycloolefin-based polymer.

9. A polarizing plate comprising two protective films having a polarizer interposed therebetween, wherein at least one of the two protective films is a film formed of a composition containing the high molecular compound according to claim 4.

10. The polarizing plate according to claim 9, wherein at least one of the two protective films is an optically anisotropic film.

11. The polarizing plate according to claim 9, wherein at least one of the two protective films is an optically compensatory film.

12. A liquid crystal display device comprising a film formed of a composition containing the high molecular compound according to claim 4.

13. The liquid crystal display device according to claim 12, wherein the film is an optically anisotropic film.

14. The liquid crystal display device according to claim 12, wherein the film is an optically compensatory film.

15. A method for producing an optically compensatory film comprising a substrate having thereon an optically anisotropic film, said method comprising (a) coating a composition containing the high molecular compound according to claim 4 on a substrate; and (b) irradiating it with polarized light from a single direction to the substrate.

16. The method for producing the optically compensatory film according to claim 15, wherein the polarized light is irradiated from a vertical direction to the substrate.

17. The method for producing the optically compensatory film according to claim 15, wherein in the (b), the polarized light is irradiated from an oblique direction to the substrate.

18. The method for producing the optically compensatory film according to claim 15, wherein the polarized light to be used for the irradiation with polarized light is p-polarized light or s-polarized light.

19. The method for producing the optically compensatory film according to claim 15, wherein the (b) is carried out at a temperature of not higher than a glass transition temperature of the high molecular compound.

20. The method for producing the optically compensatory film according to claim 15, further comprising (c) thermally treating the substrate and/or the composition after the (b).
